(19) United States
(12) Patent Application Publication
(10) Pub. No.: US 2010/0233819 A1
(43) Pub. Date: Sep. 16, 2010

(54) ROBO: A NOVEL FAMILY OF POLYPEPTIDES AND NUCLEIC ACIDS

(75) Inventors: COREY S. GOODMAN, BERKELEY, CA (US); THOMAS KIDD, BERKELEY, CA (US); KEVIN J. MITCHELL, BERKELEY, CA (US); GUY TEAR, LONDON (GB)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: THE REGENTS OF THE UNIVERSITY OF CALIFORNIA, OAKLAND, CA (US)

(21) Appl. No.: 12/401,575

(22) Filed: Mar. 10, 2009

Related U.S. Application Data
(63) Continuation of application No. 10/826,812, filed on Apr. 16, 2004, now abandoned.
Application No. 10/826,812 is a continuation of application No. 08/971,172, filed on Nov. 14, 1997, now abandoned.
(60) Provisional application No. 60/062,921, filed on Oct. 20, 1997.

(51) Int. Cl. G01N 33/68; C07K 16/00; C07K 14/435; C07K 7/06; C07K 7/08; C12N 15/12

(52) U.S. Cl. 436/86; 530/387.9; 530/391.3; 530/387.3; 530/324; 530/327; 530/328; 530/326; 536/23.5

(57) ABSTRACT

Robo1 and Robo2 polypeptides may be produced recombinantly from transformed host cells from the disclosed Robo encoding nucleic acids or purified from human cells. The invention provides isolated Robo hybridization probes and primers capable of specifically hybridizing with the disclosed Robo genes, Robo-specific binding agents such as specific antibodies, and methods of making and using the subject compositions in diagnosis, therapy and in the biopharmaceutical industry.

### CROSS-REFERENCES TO RELATED APPLICATIONS

This application is a continuation of U.S. patent application Ser. No. 10/826,812 filed Apr. 16, 2004, which is a continuation of U.S. patent application Ser. No. 08/971,172 filed Nov. 14, 1997 (now abandoned), which claims benefit of U.S. provisional application No. 60/062,921 filed Oct. 20, 1997, which applications are herein incorporated by reference.

### STATEMENT AS TO RIGHTS TO INVENTIONS MADE UNDER FEDERALLY SPONSORED RESEARCH OR DEVELOPMENT

This invention was made with government support under Grant Number NS18366 awarded by the NIH. The government has certain rights in the invention.

### INTRODUCTION

1. Field of the Invention

The field of this invention is proteins involved in nerve cell guidance.

2. Background

Bilaterally symmetric nervous systems, such as those found in insects and vertebrates, have special midline structures that establish a partition between the two mirror image halves. Axons that link the two sides of the nervous system project toward and across the midline, forming axon commissures. These commissural axons project toward the midline, at least in part, by responding to long-range chemoattractants emanating from the midline. One important class of midline chemoattractants are the netrins (Serafini et al., 1994; Kennedy et al., 1994), guidance signals whose structure, function, and midline expression is evolutionarily conserved from nematodes and fruit flies to vertebrates (Hedgecock et al., 1990; Wadsworth et al., 1996; Mitchell et al., 1996; Harris et al., 1996). The attractive actions of netrins appear to be mediated by growth cone receptors of the DCC subfamily of the immunoglobulin (Ig) superfamily (Keino-Masu et al., 1996; Chan et al., 1996; Kolodziej et al., 1996).

The midline also provides important short-range guidance signals. This is best illustrated by considering the different classes of axon projections in the spinal cord of vertebrates or the nerve cord of insects. Although some growth cones extend away from the midline, most extend towards or along the midline during some segment of their trajectory. Certain classes of growth cones either extend towards the midline or longitudinally along it and yet never cross it. Most growth cones (˜90% in the Drosophila CNS), however, do cross the midline. After crossing, the majority of these growth cones turn to project longitudinally, growing along or near the midline. Interestingly, these axons never cross the midline again, despite navigating in the vicinity of other axons that continue to cross.

What midline signals and growth cone receptors control whether growth cones do or do not cross the midline? After crossing once, what mechanism prevents these growth cones from crossing again? Studies in the chick (Stoeckli and Landmesser, 1995; Stoeckli et al., 1997) and grasshopper (Myers and Bastiani, 1993) embryos have led to the suggestion that the midline contains a contact-mediated repellent, and that commissural growth cones must overcome this repellent to cross the midline. For example, this notion that the midline can be repulsive even to growth cones that cross it is supported by time-lapse imaging of the first commissural growth cone in the grasshopper embryo. On contacting the midline, this growth cone often abruptly retracts, although ultimately it overcomes the repulsion and crosses the midline.

One approach to find the genes encoding the components of such a midline guidance system is to screen for mutations in which either too many or too few axons cross the midline. Such a large-scale mutant screen was previously conducted in Drosophila and led to the identification of two key mutations: commissureless (comm) and roundabout (robo) (Seeger et al., 1993; reviewed by Tear et al., 1993). In comm mutant embryos, commissural growth cones initially orient toward the midline but then fail to cross it and instead recoil and extend on their own side. comm encodes a novel surface protein expressed on midline cells. As commissural growth cones contact and traverse the CNS midline, Comm protein is apparently transferred from midline cells to commissural axons (Tear et al., 1996). In robo mutant embryos, many growth cones that normally extend only on their own side instead now project across the midline, and axons that normally cross the midline only once instead appear to cross and recross multiple times (Seeger et al, 1993; Kidd et al., 1997). Double mutants of comm and robo display a robo-like phenotype.

Here we disclose the characterization of robo across animal species. robo encodes a new class of guidance receptor with 5 Ig domains, 3 fibronectin (FN) type III domains, a transmembrane domain, and a long cytoplasmic domain. Robo defines a new subfamily of Ig superfamily proteins that is highly conserved from fruit flies to mammals. The results of protein expression and transgenic rescue experiments indicate that Robo functions as the gatekeeper controlling midline crossing and that Robo responds to an unknown midline repellent.

### SUMMARY OF THE INVENTION

The invention provides methods and compositions relating to Robo1 and Robo2, collectively Robo) polypeptides, related nucleic acids, polypeptide domains thereof having Robo-specific structure and activity, and modulators of Robo function. Robo polypeptides can regulate cell, especially nerve cell, function and morphology. The polypeptides may be produced recombinantly from transformed host cells from the subject Robo polypeptide encoding nucleic acids or purified from mammalian cells. The invention provides isolated Robo hybridization probes and primers capable of specifically hybridizing with natural Robo genes, Robo-specific binding agents such as specific antibodies, and methods of making and using the subject compositions in diagnosis (e.g. genetic hybridization screens for Robo transcripts), therapy (e.g. Robo inhibitors to promote nerve cell growth) and in the biopharmaceutical industry (e.g. as immunogens, reagents for isolating Robo genes and polypeptides, reagents for screening chemical libraries for lead pharmacological agents, etc.).

### BRIEF DESCRIPTION OF THE FIGURES

FIG. 1 Organization of the roundabout Genomic Locus

(A) Cosmid chromosome walk through the 58F/59A region of the 2nd chromosome. The position of deficiency breakpoints within the cosmids used are shown in the top two rows. Identified transcripts from the walk are shown below the cosmids. The 12-1 transcript corresponds to the robo gene; the direction of transcription is distal to proximal. The location of the 16 kb XbaI genomic rescue fragment is indicated below.

(B) Position and size of introns within the robo transcript. Coding sequence is indicated by the thicker part of the line. Introns are represented by gaps. The transcript is shown 3′-5′ to reflect its orientation in (A).

FIG. 2 Structure of Robo Protein

Schematic of the structure of Drosophila Robo protein. The position of the Immunoglobulin (Ig), fibronectin (FN) and transmembrane (TM) domains and the amino acid substitution in robo⁶ are shown. Percent amino acid identity between Drosophila Robo 1 and Human Robo 1 is indicated for each domain.

### DETAILED DESCRIPTION OF THE INVENTION

The nucleotide sequences of exemplary natural cDNAs encoding drosophila 1, drosophila 2, C. elegans, human 1, human 2 and mouse 1 Robo polypeptides are shown as SEQ ID NOS:1, 3, 5, 7, 9 and 11, respectively, and the full conceptual translates are shown as SEQ ID NOS:2, 4, 6, 8, 10 and 12. The Robo polypeptides of the invention include incomplete translates of SEQ ID NOS:1, 3, 5, 7, 9 and 11 and deletion mutants of SEQ ID NOS:2, 4, 6, 8, 10 and 12, which translates and deletion mutants have Robo-specific amino acid sequence, binding specificity or function. Preferred translates/deletion mutants comprise at least a 6, preferably at least an 8, more preferably at least a 32, most preferably at least a 64 residue domain of the translates. In a particular embodiment, the deletion mutants comprise one or more structural/functional Robo immunoglobulin, fibronectin or cytoplasmic motif domains described herein. For example, soluble forms of the disclosed Robo polypeptides which comprise one or more Robo IG domains, and especially fusions of two or more Robo IG domains, particularly fusions of IG#1 and #2, provide competitive inhibitors of Robo-mediated signaling. Exemplary such deletion mutants and recombined deletion mutant fusions include human Robo 1 (SEQ ID NO:8) residues 1-67; 68-167; 168-259; 260-350; 351-451; 1-167; 1-259; 1-350; 1-451; 68-259; 1-67 joined to 168-259; and 1-67 joined to 260-451.

Other deletion mutants provide Robo-specific antigens and/or immunogens, especially when coupled to carrier proteins as described below. Generic Robo-specific peptides are readily apparent as conserved regions in the aligned Robo polypeptide sequences of Table 1.

TABLE 1

Sequence Alignment of Robo Family Members: The complete amino acid
alignment of the predicted Robo proteins encoded by drosophila robo 1
(D1, SEQ ID NO: 2) and Human robo 1 (H1, SEQ ID NO: 8) are shown. The
extracellular domain of C. elegans robo (CE, SEQ ID NO: 6; Sax-3;
Zallen et al., 1997), the extracellular domain of Drosophila robo 2
(D2, SEQ ID NO: 4), and partial sequence of Human robo 2 (H2, SEQ ID
NO: 10) are also aligned. The D2 sequence was predicted by the gene-
finder program Grail. The position of immunoglobulin domains (Ig),
fibronectin domains (FN), the transmembrane domain (TM), and conserved
cytoplasmic motifs are indicated. The extracellular domain of rat robo
1 is nearly identical to H1.

| | | |
|---|---|---|
| mH.............PMHpENHAIaRSTSTTNNPSrsRSSRMWLlpAWLLLVLVASNGLP | 47 | D1 |
| m.FNRKTLlCTi.llVlQA..............vIrsFCEDASNlA.............. | 30 | CE |
| mKWKHVPFlVMiSllSlSpNHLFLaQLIPDPEDvErG.NDHGTPIpTSDNDDNSLGYTGS | 59 | H1 |
| >IG #1 | | |
| AVrGQYQSpriiehpTdlvvKknepatlnckVegKpEptiewfkdgepvStn..EKKshr | 105 | D1 |
| GENpriiehpMdTTvPknDpFtFncQaegNptptiQwfkdgRELKt...dTGshr | | D2 |
| ........pViiehpIdVvvsRgSpatlncGaK.PStAKiTwykdgQpvItnkEQVNshr | 81 | CE |
| RLrQEDFPpriVehpSdlIvskgepatlnckaegRptptiewykGgeRvEtDkDdPRshr | 119 | H1 |
| >IG #2 | | |
| VQFKDgAlffYriMQgkkeQ..dGgEywcvaknRVgQaysrHaslqIavlrddfrvepKd | 163 | D1 |
| iMlpAgGlfflkvIhSrReS..dagTywcEakneFgVaRsrnaTlqvavlrdEfrLepAN | | D2 |
| iVlDTgslfLlkvNSgkNGKDSdagAyYcvaSneHgeVKsNEGslKLaMlrEdfrvRpRT | 141 | CE |
| MLlpSgslfflriVhgrkSRP.dEgVyVcvaRnYLgeaysHnaslEvaIlrddfrQNpSd | 178 | H1 |
| trvaKgeTallecgppKgIpeptLIwIkdgVplddLKAmSFGASSrVrivdggnlLiSNv | 223 | D1 |
| trvaQgeValmecgAprgSpepQiswrkNgQTlNL......VGNKririvdggnlAiQEA | | D2 |
| vQALGgeMavlecSpprgFpepVVswrkdDKElRI.QDmP.....rYTLHSDgnlIiDPv | 195 | CE |
| vMvaVgePavmecQpprgHpeptiswKkdgSpldd.......KDEri.TIRggKlMiTYT | 230 | H1 |
| >IG #3 | | |
| EPIdEgNyKcIaQnLvgtresSYaKlIvQvkpYfMkepkdgVMLYgQTaTfHcSvggdpP | 283 | D1 |
| rQsdDgRyqcvVKnVvgtresATaFlKvHvrpFLIRGpQnqtAVvgSsvVfQcrIggdpL | | D2 |
| DRsdSgTyqcvaNnmvgerVsNPaRlSvFekpKfEQepkdMtvDvgAAvLfDcrvTgdpQ | 255 | CE |
| rKsdAgKyVcvGTnmvgeresEVaElTvLerpSfVkRpSnLAvTvDDsaEfKcEARgdpV | 290 | H1 |
| pKvlwkk..EEgnIpvsrA..........RiLHdEKslEiSNItpTdegTyvceaHnNvg | 331 | D1 |
| pDvlwrrTASGgnmpLRKFSWLHSASGRVHVl.EdrslkLDDvtLEdmgeytceaDnAvg | | D2 |
| pQITwkr..KNEPmpvTra..........YiAKdNrGlRiERvQpSdegeyvcYaRnPAg | 303 | CE |
| pTvRwrk..DDgELpKsrY..........Ei.RddHTlkiRKvtAGdmgSytcVaEnMvg | 337 | H1 |
| >IG #4 | | |
| QiSaRaSlIvhappNfTKrpSnKKvGlNgVvQLPcMaSgnpPpSvfwTkegVSTlMfpn. | 388 | D1 |
| GiTaTGIltvhappKfvIrpKnqLvEIgDEvLfecQaNgHpRpTLYwsVegNSSllLpGy | | D2 |
| TLeasaHlRvqappSfQTkpAdqSvPAggtAtfecTLVgQpSpaYfwskegQqDllfpsy | 363 | CE |
| KAeasaTltvqEppHfvVkpRdqVvalgrtvtfQceaTgnpqpaIfwRRegsqnllf.sy | 396 | H1 |
| gIvaQgrtvtfPceTKgnpqpavfwQkegsqnllfpn. | | H2 |
| ...SsHGrQYvAADgtlQitDvrqedegyyv.cSaFSvvDssTVrVFlQvSS..vD.... | 440 | D1 |
| RDGRMEVTLTPEGRSVlSiARFAredSgKVvTcNalnAvgsVSsrTVVSvDt..QF.... | | D2 |
| VSADGRTK..vsptgtltiEEvrqVdegAyv.cAGMnSagsslskaAlKvttKAvTGNTP | 420 | CE |
| qpPQsSsrFsysQtgdltitnvqrsdVgyyi.cqTlnvagsiITkaYlevtd..vIA... | 450 | H1 |
| qpQQPNsrCsysptgdltitnIgrsdAgyyi.cqalTvagsilAkaQlevtd..vLT... | | H2 |
| >IG #5 | | |
| erpppiiQIgpAnqtlpKgsVaTlpcratgNpSpRiKwFHdgHAvQA.GNRYSi.igG.. | 496 | D1 |
| eLpppiieqgpvnqtlpvKsIVvlpcrTLgTpvpQVswYLdgIpidVqEHERrNLsDA.. | | D2 |
| AKpppTieHgHQnqtlMvgsSaIlpcQaSgKpTpGiswlRdgLpidITd..sri.sqHST | 477 | CE |
| drpppViRqgpvnqtVavdgtFvlScVatgSpvpTiLwRkdgVLvSTqd..sriK.qLeN | 507 | H1 |
| drpppiiLqgpAnqtlavdgtaLcKcKatgDpLpViswlkEgFTFPGRd..PrATiq.eQ | | H2 |
| >FN #1 | | |
| SslRVDdlq.lsdSgtytciasGeRgeTswAaTltveKpgs..TSLHraAdpstypAppg | 553 | D1 |
| gAlTiSdlqrHEdEgLytcvasnRNgKsswsGylRLDTptNpNiKfFrapElstypgppg | | D2 |
| gslHiAdl.kKPdtgVytciaKneDgestwsaSltveDHtsN.AqfVrMpdpsNFpsSpT | 535 | CE |
| gvlqiR.YAklGdtgRytciasTPsgeatwsayIEvQeFgVp.VqPPrPTdpNLIpsAps | 565 | H1 |
| gTlgiKNl.rIsdtgtytcvaTSSsgeaswsaVlDvTeSgAT.i..SKNYdlsDLpgpps | | H2 |
| TpKvLnvsrtsISlRwAKSqEKPGAVgpIi.gyTVeyfspdlQTgwIVAaHrvGDtQVti | 612 | D1 |
| kpqMvEKGEnsvtlsw...TRSNKVggSSLVgyVieMfGKNETDgwVAvGTrvQNttFtQ | | D2 |
| QpIIvnvtDtEvElHw...NAPSTsgaGpitgyiiQyYspdlgQTwFNIPDYvAStEyRi | 592 | CE |
| kpEvtdvsrnTvtlsw...qpNLNsgaTp.tSyiieafsHASgSswqtvaENvktEtSAi | 621 | H1 |
| kpqvtdvtKnsvtlsw...qpGTPGTLpA.SAyiieafsQSVSNswqtvaNHvkttLytV | | H2 |
| >FN #2 | | |
| SglTpgtsyVflvraenTQgisvpsGLsNViktIEA....DfDAASANdlsAarT.llTg | 667 | D1 |
| TglLpgVNyFfliraenSHgLsLpsPMsEpitVGTR....YfNS..gLdlsEarASllsg | | D2 |
| kglkpSHsyMfViraenEkgiGTpsVSsALvttSKPAAQVAlSDKNKMdMAIaEKRlTsE | 652 | CE |
| kglkpnAiylflvraAnAYgisDpsqIsDpvktQDV.....lPTSQgVdHKQVQRE.lGN | 675 | H1 |
| RglRpntiylfMvraInPkV.svT.q | | H2 |
| KSvelIDasAinAsavrlEwMLHvSADEkyvegLRiHyK..DaSVPSAQYHSITvMDAsa | 725 | D1 |
| DvvelSnasvVDstsMKlTwQI...INGkyvegFyVYArQLpNPLNTKyRMLTILNGGGa | | D2 |
| QLIKlEEVKTinstavrlFwKKR..KLEELiDgyyiKWrGPpRTNDNQyVN...vTSpsT | 707 | CE |
| AvLHlHnPTvLSsssIEVHwT..vDQQSQyiQgyKiLyrPSGaNHGESDWLVFEvRTpAK | 733 | H1 |
| >FN #3 | | |
| esFvvGnlKkytKyeffLTpf...fETiegQpsnskTaltYedvpsappDNIQiGmYn.. | 780 | D1 |
| SsCTiTGlVQytLyeffIVpf...YKsVegKpsnsRIaRtledvpsEApYgMEALLln.. | | D2 |
| eNYvvSnlMPFtnyeffVIpYHSGVHsiHgapsnsMDVltAeAPpsLppEDvRiRmlnL. | 766 | CE |
| NsVviPDlRkGVnyeIKARpf...fNEFQgaDsEIkFaKtleEApsappQgvTVSKNDGN | 790 | H1 |
| QtaGWvRwTpppSQHHngNlYgykiEVSAgnTM.....KVlAnMtLnaTtTsvLlNnltt | 835 | D1 |
| SSaVFLKwkapELKDRHgVlLNyH.vivRgIDtAHNFSRIlTnVtIdaASPTLvlAnitE | | D2 |
| .tTLRIswkapKAdGIngIlKgFQiviv.gQAPNNNR.....nItTnERAAsvTlFHlVt | 819 | CE |
| GtaILvswQpppEdTQngMVQEykV.WCLgnEtR.....YHInKtVdGStFsvvIPFlVP | 844 | H1 |
| gAVysvrLNSFtKagDgpysKpISlFMdpTHHVHPpRAHPsGTHDGRHEGqDLTYHNNgN | 895 | D1 |
| gVMyTvGvaaGNnagvgpyCVpATlRldpITKRLDpFINQRDHVND.............. | | D2 |
| gMTyKIrvAARSnGgvgv..........ShgTSEVIMNqDTlEKHL.AAQqENESFLYgL | 868 | CE |
| gIRysvEvaaStGagSgvKsEpQFIQldAhgNPVSpEDqVslAQQI.............. | 890 | H1 |
| > TM < | | |
| iPPGDINPTTHKKTTdYlSGpwLMViVCiVlLvlVisAAIsM.vyFkrkhQmTKElGHLS | 954 | D1 |
| ................vlTqpwFIiiLgAilavlMLs..fGAMvFVkrkhMm..MkQsAL | | D2 |
| iNK..............SHVpVIViVaILiIFvViiIAY.CYwRNS.rNSD...gkDRSF | 909 | CE |
| ..............SdvVKqp..AFiagiGAaCWiiLMVfsIwLyRHrkKR..NglTsTY | 932 | H1 |
| VVSDNEIT.......................AlniNSKESL.wIDHHRGwRTADTDKD.. | 988 | D1 |
| AGIRKVPSFTFTPTVTYQRGGEAVSSGGRPGLlniSEPAAQPwLAD..TwPNTGNNHNDC | 990 | H1 |
| ........SgLsEsKlLSHVNSSQ..SnynnS..........DGGtDyAEvd....TRNL | 1024 | D1 |
| SISCCTAGNgNsDsNlTTYSRPADCIAnynnQLDNKQTNLMLPEStVyGDvdLSNKINEM | 1050 | H1 |
| CYTOPLASMIC MOTIF #1 | | |
| TtfYNCR.......KSPDNptpyattMIiGTS........sSETCTkT.TSISADkDSGT | 1068 | D1 |
| KtfNSPNLKDGRFVNPSGQptpyattQLiQSNLSNNMNNGsGDSGEkHWKPLGQQkQEVA | 1110 | H1 |
| HSPyS........DAFAGQVPAVpVV..KSNyLqYPVEP..................... | 1097 | D1 |
| PVQyNIVEQNKLNKDYRANDTVPpTIPYNQSyDqNTGGSYNSSDRGSSTSGSQGHKKGAR | 1170 | H1 |
| CYTOPLASMIC MOTIF #2 | | |
| .........InwSEFlppppEhppp...sSTy......GyAgGSp............... | 1124 | D1 |
| TPKVPKQGGMnwADLlppppAhpppHSNsEEyNISVDESyDqEMpCPVPPARMYLQQDEL | 1230 | H1 |
| ..eSSRKSSKSAGSgISTNQSILNAsIHsSSSGGFsAWGVSPQYAVAcp........... | 1171 | D1 |
| EEeEDERGPTPPVRgAASSPAAVSYsHQsTATLTPsPQEELQPMLQDcpEETGHMQHQPD | 1290 | H1 |
| ................pENVy...sNpl.....SAVAGGTQNRYQITPTNQHPPQl.... | 1203 | D1 |
| RRRQPVSPPPPPRPISpPHTyGYIsGplVSDMDTDAPEEEEDEADMEVAKMQTRRlLLRG | 1350 | H1 |
| ....paY................FATTGPGGAVPPNHLP.............faTQRHaa | 1230 | D1 |
| LEQTpaSSVGDLESSVTGSMINGWGSASEEDNISSGRSSVSSSDGSFFTDADfaQAVAaa | 1410 | H1 |
| SeyQaglNAar................cAQSRACNsCdALATPSPmq............. | 1261 | D1 |
| Aey.aglKVarRQMQDAAGRRHFHASQcPRPTSPVsTdSNMSAAVmqKTRPAKKLKHQPG | 1469 | H1 |
| CYTOPLASMIC MOTIF #3 | | |
| ...........ppppvpVpEGWYQPVHPNSH.PMHpTS.SNHQIYQCSSECsDHSRSsQS | 1307 | D1 |
| HLRRETYTDDLppppvpPpAIKSPTAQSKTQLEVRpVVVPKLPSMDARTDRsSDRKGsSY | 1529 | H1 |
| HKrQL.................QLEeHGSSAkQrgGHHRRrA.pVVQPCMESeN......ENM | | D1 |
| KGrEVLDGRQVVDMRTNPGDPREAQeQQNDGkGrgNKAAKrDLpPAKTHLIQeDILPYCRPTF | | H1 |
| LAEYEQrQYTsDCCNssrEGDTC..........SCSeGSCL..yAeAgePAPRQMTAKNT | 1395 | D1 |
| PTSNNPrDPSsSSSMssrGSGSRQREQANVGRRNIAeMQVLGGy.eRgeDNNEELEETES | 1651 | H1 |

Exemplary such Robo specific immunogenic and/or antigenic peptides are shown in Table 2.

TABLE 2
Immunogenic Robo polypeptides eliciting Robo-specific
rabbit polyclonal antibody: Robo polyeptide-KLH conjugates
immunized per protocol described below.

| Robo Polypetide, Sequence | Immunogenicity |
|---|---|
| SEQ ID NO: 2, residues 68-77 | +++ |
| SEQ ID NO: 2, residues 79-94 | +++ |
| SEQ ID NO: 2, residues 95-103 | +++ |
| SEQ ID NO: 2, residues 122-129 | +++ |
| SEQ ID NO: 2, residues 165-176 | +++ |
| SEQ ID NO: 2, residues 181-191 | +++ |
| SEQ ID NO: 2, residues 193-204 | +++ |
| SEQ ID NO: 2, residues 244-251 | +++ |
| SEQ ID NO: 2, residues 274-290 | +++ |
| SEQ ID NO: 2, residues 322-331 | +++ |
| SEQ ID NO: 2, residues 339-347 | +++ |
| SEQ ID NO: 2, residues 407-417 | +++ |
| SEQ ID NO: 2, residues 441-451 | +++ |
| SEQ ID NO: 2, residues 453-474 | +++ |
| SEQ ID NO: 2, residues 502-516 | +++ |
| SEQ ID NO: 2, residues 541-553 | +++ |
| SEQ ID NO: 2, residues 617-629 | +++ |

In addition, species-specific antigenic and/or immunogenic peptides are readily apparent as diverged extracellular or cytosolic regions in Table 1. Exemplary such human specific peptides are shown in Table 3.

TABLE 3
Immunogenic Robo polypeptides eliciting human Robo-specific rabbit
polyclonal antibody: Robo polyeptide-KLH conjugates immunized
per protocol described below (some antibodies show cross-reactivity
with corresponding mouse/rat Robo polypeptides).

| Robo Polypetide, Sequence | Immunogenicity |
|---|---|
| SEQ ID NO: 8, residues 1-12 | +++ |
| SEQ ID NO: 8, residues 18-28 | +++ |
| SEQ ID NO: 8, residues 31-40 | +++ |
| SEQ ID NO: 8, residues 45-65 | +++ |
| SEQ ID NO: 8, residues 106-116 | +++ |
| SEQ ID NO: 8, residues 137-145 | +++ |
| SEQ ID NO: 8, residues 174-184 | +++ |
| SEQ ID NO: 8, residues 214-230 | +++ |
| SEQ ID NO: 8, residues 274-286 | +++ |
| SEQ ID NO: 8, residues 314-324 | +++ |
| SEQ ID NO: 8, residues 399-412 | +++ |
| SEQ ID NO: 8, residues 496-507 | +++ |
| SEQ ID NO: 8, residues 548-565 | +++ |
| SEQ ID NO: 8, residues 599-611 | +++ |
| SEQ ID NO: 8, residues 660-671 | +++ |
| SEQ ID NO: 8, residues 717-730 | +++ |
| SEQ ID NO: 8, residues 780-791 | +++ |
| SEQ ID NO: 8; residues 835-847 | +++ |
| SEQ ID NO: 8, residues 877-891 | +++ |
| SEQ ID NO: 8, residues 930-942 | +++ |
| SEQ ID NO: 8, residues 981-998 | +++ |
| SEQ ID NO: 8, residues 1040-1051 | +++ |
| SEQ ID NO: 8, residues 1080-1090 | +++ |
| SEQ ID NO: 8, residues 1154-1168 | +++ |
| SEQ ID NO: 8, residues 1215-1231 | +++ |
| SEQ ID NO: 8, residues 1278-1302 | +++ |
| SEQ ID NO: 8, residues 1378-1400 | +++ |
| SEQ ID NO: 8, residues 1460-1469 | +++ |
| SEQ ID NO: 8, residues 1497-1519 | +++ |
| SEQ ID NO: 8, residues 1606-1626 | +++ |
| SEQ ID NO: 8, residues 1639-1651 | +++ |
| SEQ ID NO: 10, residues 5-16 | +++ |
| SEQ ID NO: 10, residues 38-47 | +++ |
| SEQ ID NO: 10, residues 83-94 | +++ |
| SEQ ID NO: 10, residues 112-125 | +++ |
| SEQ ID NO: 10, residues 168-180 | +++ |
| SEQ ID NO: 10, residues 195-209 | +++ |
| SEQ ID NO: 10, residues 222-235 | +++ |
| SEQ ID NO: 10, residues 241-254 | +++ |

In a particular embodiment, expressed sequence tags EST;yu23d11, Accession #H77734 and EST;yq76e12, Accession #H52936, as well as peptides conceptually encoded thereby, are not within the scope of the present invention (Tables 4 and 5). In a particular embodiment, the subject Robo polypeptides exclude the corresponding regions of the disclosed natural human Robo I polypeptide, i.e. SEQ ID NO:8, residues 168-217 and SEQ ID NO:8, residues 1316-1485.

TABLE 4

EST: yu23d11 sequences compared to H-Robo1. yu23d11 refers to
the fragment of DNA which was sequenced. The fragment was
sequenced from both ends generating the following two
sequences: H77734 and H77733. yu23d11 is an unspliced cDNA.
Only bases 59-215 match the coding sequence of H-Robo1
(502-651). The remaining bases are intronic. No bases of
H77733 match the coding sequence of H-Robo1.

| | |
|---|---|
| LRDDFRQNPSDVMVAVGEPAVMECQPPRGHPEPTISWKKDGSPLDDKDER | H-Robo1 |
| LRDDFRQKPSDVMVAVGEPAVMECQPPRGHPEPTISWKKDGSPLDDKDER | EST H77734 |

There is an error in the sequence, a T to G change which results in the amino acid N being replaced by K. The sequence is shown below and has been reversed for clarity:

| | |
|---|---|
| TACTTCGGGATGACTTCAGACAAAAACCTTCGGATGTCATGGTTGCAGTA | H-Robo1 |
| TACTTCGGGATGACTTCAGACAAAACCCTTCGGATGTCATGGTTGCAGTA | EST H77734 |
| L R D D F R Q K P S D V M V A V | |
| N | |

TABLE 5
EST: yq76e12 sequences compared to H-Robo1.
yq76e12 refers to the fragment of DNA which was
sequenced. The fragment was sequenced from both
ends generating the following two sequences:
H52936 and H52937 (the latter has been reversed
for clarity). The sequences can be seen to overlap
in the middle. A gap indicates a frameshift error.
Note that errors only occur in one sequence at any
one position.

| | |
|---|---|
| GPLVSDMDTDAPEEEEDEADMEVAKMQTRRLLLRGLEQTPASSV | H- |
| | Robo1 |
| GPLVSDMDTDAPEEEEDEADMEVAKMQT.RLLLRGLEQTPASSV | EST |
| | H52936 |
| GDLESSVTGSMINGWGSASEEDNISSGRSSVSSSDGSFFTDADF | H- |
| | Robo1 |
| GDLESSVTGSMINGWGSASEEDNISSGRSSVSSSDGSFFTDADF | EST |
| | H52936 |
| AQAVAAA AEYAGLKVARRQMQDA AGR RHFH AS QC PRPT | H- |
| | Robo1 |
| AQAVAAA AEYAGLKVARRQMQDA AGR RHFH AF QC PRPT | EST |
| | H52936 |
| ?AAT A?YAGLKVARRQMRDA AGR RHFH AS QC PRPT | EST |
| | H52937 |
| SPVSTDSNMSAAVMQKTRPAKKLKHQPGHLRRETYTDDLPPPPV | H- |
| | Robo1 |
| SPVFTDSNM | EST |
| | H52936 |
| SPVSTDSNMSAAVMQKTRPAKKLKHQPGHLRRETYTDDLPPPPV | EST |
| | H52937 |
| PPPAIKSPTAQSKTQLEVRPVVVPKLPSMDARTDK | H- |
| | Robo1 |
| PPPAIKSPTAQSKTQLEVRPVVVPKLPSMDARTDK | EST |
| | H52937 |

The subject domains provide Robo domain specific activity or function, such as Robo-specific cell, especially neuron modulating or modulating inhibitory activity, Robo-ligand-binding or binding inhibitory activity. Robo-specific activity or function may be determined by convenient in vitro, cell-based, or in vivo assays: e.g. in vitro binding assays, cell culture assays, in animals (e.g. gene therapy, transgenics, etc.), etc. Binding assays encompass any assay where the molecular interaction of a Robo polypeptide with a binding target is evaluated. The binding target may be a natural intracellular binding target, a Robo regulating protein or other regulator that directly modulates Robo activity or its localization; or non-natural binding target such as a specific immune protein such as an antibody, or a Robo specific agent such as those identified in screening assays such as described below. Robo-binding specificity may be assayed by binding equilibrium constants (usually at least about 10⁷ M⁻¹, preferably at least about 10⁸ M⁻¹, more preferably at least about 10⁹ M⁻¹), by the ability of the subject polypeptide to function as negative mutants in Robo-expressing cells, to elicit Robo specific antibody in a heterologous host (e.g a rodent or rabbit), etc.

The claimed Robo polypeptides are isolated or pure: an “isolated” polypeptide is unaccompanied by at least some of the material with which it is associated in its natural state, preferably constituting at least about 0.5%, and more preferably at least about 5% by weight of the total polypeptide in a given sample and a pure polypeptide constitutes at least about 90%, and preferably at least about 99% by weight of the total polypeptide in a given sample. A polypeptide, as used herein, is a polymer of amino acids, generally at least 6 residues, preferably at least about 10 residues, more preferably at least about 25 residues, most preferably at least about 50 residues in length. The Robo polypeptides and polypeptide domains may be synthesized, produced by recombinant technology, or purified from mammalian, preferably human cells. A wide variety of molecular and biochemical methods are available for biochemical synthesis, molecular expression and purification of the subject compositions, see e.g. Molecular Cloning, A Laboratory Manual (Sambrook, et al. Cold Spring Harbor Laboratory), Current Protocols in Molecular Biology (Eds. Ausubel, et al., Greene Publ. Assoc., Wiley-Interscience, NY) or that are otherwise known in the art.

The invention provides binding agents specific to the claimed Robo polypeptides, including natural intracellular binding targets, etc., methods of identifying and making such agents, and their use in diagnosis, therapy and pharmaceutical development. For example, specific binding agents are useful in a variety of diagnostic and therapeutic applications, especially where pathology, wound repair incompetency or prognosis is associated with improper or undesirable axon outgrowth, orientation or inhibition thereof. Novel Robo-specific binding agents include Robo-specific receptors, such as somatically recombined polypeptide receptors like specific antibodies or T-cell antigen receptors (see, e.g. Harlow and Lane (1988) Antibodies, A Laboratory Manual, Cold Spring Harbor Laboratory), natural intracellular binding agents identified with assays such as one-, two- and three-hybrid screens, non-natural intracellular binding agents identified in screens of chemical libraries such as described below, etc. Agents of particular interest modulate Robo function.

In a particular embodiment, the subject polypeptides are used to generate Robo- or human Robo-specific antibodies. For example, the Robo- and human Robo-specific peptides described above are covalently coupled to keyhole limpet antigen (KLH) and the conjugate is emulsified in Freunds complete adjuvant. Laboratory rabbits are immunized according to conventional protocol and bled. The presence of Robo-specific antibodies is assayed by solid phase immunosorbant assays using immobilized Robo polypeptides of SEQ ID NO:2, 4, 6, 8, 10 or 12. Human Robo-specific antibodies are characterized as uncross-reactive with non-human Robo polypeptides (SEQ ID NOS:2, 4, 6 and 12).

Accordingly, the invention provides methods for modulating cell function comprising the step of modulating Robo activity, e.g. by contacting the cell with a Robo inhibitor, e.g. inhibitory Robo deletion mutants, Robo-specific antibodies, etc. (supra). The target cell may reside in culture or in situ, i.e. within the natural host. The inhibitor may be provided in any convenient way, including by (i) intracellular expression from a recombinant nucleic acid or (ii) exogenous contacting of the cell. For many in situ applications, the compositions are added to a retained physiological fluid such as blood or synovial fluid. For CNS administration, a variety of techniques are available for promoting transfer of the therapeutic across the blood brain barrier including disruption by surgery or injection, drugs which transiently open adhesion contact between CNS vasculature endothelial cells, and compounds which facilitate translocation through such cells. Robo polypeptide inhibitors may also be amenable to direct injection or infusion, topical, intratracheal/nasal administration e.g. through aerosol, intraocularly, or within/on implants e.g. fibers e.g. collagen, osmotic pumps, grafts comprising appropriately transformed cells, etc. A particular method of administration involves coating, embedding or derivatizing fibers, such as collagen fibers, protein polymers, etc. with therapeutic proteins. Other useful approaches are described in Otto et al. (1989) J Neuroscience Research 22, 83-91 and Otto and Unsicker (1990) J Neuroscience 10, 1912-1921. Generally, the amount administered will be empirically determined, typically in the range of about 10 to 1000 mg/kg of the recipient and the concentration will generally be in the range of about 50 to 500 mg/ml in the dose administered. Other additives may be included, such as stabilizers, bactericides, etc. will be present in conventional amounts. For diagnostic uses, the inhibitors or other Robo binding agents are frequently labeled, such as with fluorescent, radioactive, chemiluminescent, or other easily detectable molecules, either conjugated directly to the binding agent or conjugated to a probe specific for the binding agent.

The amino acid sequences of the disclosed Robo polypeptides are used to back-translate Robo polypeptide-encoding nucleic acids optimized for selected expression systems (Holler et al. (1993) Gene 136, 323-328; Martin et al. (1995) Gene 154, 150-166) or used to generate degenerate oligonucleotide primers and probes for use in the isolation of natural Robo-encoding nucleic acid sequences (“GCG” software, Genetics Computer Group, Inc, Madison Wis.). Robo-encoding nucleic acids used in Robo-expression vectors and incorporated into recombinant host cells, e.g. for expression and screening, transgenic animals, e.g. for functional studies such as the efficacy of candidate drugs for disease associated with Robo-modulated cell function, etc.

The invention also provides nucleic acid hybridization probes (Tables 6, 7) and replication/amplification primers (Tables 7, 8) having a Robo cDNA specific sequence comprising SEQ ID NO:1, 3, 5, 7, 9 or 11 and sufficient to effect specific hybridization thereto (i.e. specifically hybridize with SEQ ID NO:1, 3, 5, 7, 9 or 11, respectively, in the presence of CDO cDNA.

TABLE 5
Hybridisation Probes for Human Roundabout 1

Immunoglobulin Domain #1
CCACCTCGCATTGTTGAACACCCTTCAGACCTGATTGTCTCAAAAGGAGA
ACCTGCAACTTTGAACTGCAAAGCTGAAGGCCGCCCCACACCCACTATTG
AATGGTACAAAGGGGGAGAGAGAGTGGAGACAGACAAAGATGACCCTCGC
TCACACCGAATGTTGCTGCCGAGTGGATCTTTATTTTTCTTACGTATAGT
ACATGGACGGAAAAGTAGACCTGATGAAGGAGTCTATGTCTGTGTAGCAA
GGAATTACCTTGGAGAGGCTGTGAGCCACAATGCATCGCTGGAAGTAGCC
ATA
Immunoglobulin Domain #2
CTTCGGGATGACTTCAGACAAAACCCTTCGGATGTCATGGTTGCAGTAGG
AGAGCCTGCAGTAATGGAATGCCAACCTCCACGAGGCCATCCTGAGCCCA
CCATTTCATGGAAGAAAGATGGCTCTCCACTGGATGATAAAGATGAAAGA
ATAACTATACGAGGAGGAAAGCTCATGATCACTTACACCCGTAAAAGTGA
CGCTGGCAAATATGTTTGTGTTGGTACCAATATGGTTGGGGAACGTGAGA
GTGAAGTAGCCGAGCTGACTGTCTT
Immunoglobulin Domain #3
AGAGAGACCATCATTTGTGAAGAGACCCAGTAACTTGGCAGTAACTGTGG
ATGACAGTGCAGAATTTAAATGTGAGGCCCGAGGTGACCCTGTACCTACA
GTACGATGGAGGAAAGATGATGGAGAGCTGCCCAAATCCAGATATGAAAT
CCGAGATGATCATACCTTGAAAATTAGGAAGGTGACAGCTGGTGACATGG
GTTCATACACTTGTGTTGCAGAAAATATGGTGGGCAAAGCTGAAGCATCT
GCTACTCTGACTGTTCAAGAACC
Immunoglobulin Domain #4
CCACATTTTGTTGTGAAACCCCGTGACCAGGTTGTTGCTTTGGGACGGAC
TGTAACTTTTCAGTGTGAAGCAACCGGAAATCCTCAACCAGCTATTTTCT
GGAGGAGAGAAGGGAGTCAGAATCTACTTTTCTCATATCAACCACCACAG
TCATCCAGCCGATTTTCAGTCTCCCAGACTGGCGACCTCACAATTACTAA
TGTCCAGCGATCTGATGTTGGTTATTACATCTGCCAGACTTTAAATGTTG
CTGGAAGCATCATCACAAAGGCATATTTGGAAGTTACAGATGTGATTGCA
Immunoglobulin Domain #5
GATCGGCCTCCCCCAGTTATTCGACAAGGTCCTGTGAATCAGACTGTAGC
CGTGGATGGCACTTTCGTCCTCAGCTGTGTGGCCACAGGCAGTCCAGTGC
CCACCATTCTGTGGAGAAAGGATGGAGTCCTCGTTTCAACCCAAGACTCT
CGAATCAAACAGTTGGAGAATGGAGTACTGCAGATCCGATATGCTAAGCT
GGGTGATACTGGTCGGTACACCTGCATTGCATCAACCCCCAGTGGTGAAG
CAACATGGAGTGCTTACATTGAAGTTCAAGAATTTG
Fibronectin Domain #1
GAGTTCCAGTTCAGCCTCCAAGACCTACTGACCCAAATTTAATCCCTAGT
GCCCCATCAAAACCTGAAGTGACAGATGTCAGCAGAAATACAGTCACATT
ATCGTGGCAACCAAATTTGAATTCAGGAGCAACTCCAACATCTTATATTA
TAGAAGCCTTCAGCCATGCATCTGGTAGCAGCTGGCAGACCGTAGCAGAG
AATGTGAAAACAGAAACATCTGCCATTAAAGGACTCAAACCTAATGCAAT
TTACCTTTTCCTTGTGAGGGCAGCTAATGCATATGGAATTAGTGATC
Fibronectin Domain #2
CAAGCCAAATATCAGATCCAGTGAAAACACAAGATGTCCTACCAACAAGT
CAGGGGGTGGACCACAAGCAGGTCCAGAGAGAGCTGGGAAATGCTGTTCT
GCACCTCCACAACCCCACCGTCCTTTCTTCCTCTTCCATCGAAGTGCACT
GGACAGTAGATCAACAGTCTCAGTATATACAAGGATATAAAATTCTCTAT
CGGCCATCTGGAGCCAACCACGGAGAATCAGACTGGTTAGTTTTTGAAGT
GAGGACGCCAGCCAAAAACAGTGTGGTAATCCCTGATCTCAGAAAGGGAG
TCAACTATGAAATTAAGGCTCGCCCTTTTTTTAATGAATTTCAAGGAGCA
G
Fibronectin Domain #3
ATAGTGAAATCAAGTTTGCCAAAACCCTGGAAGAAGCACCCAGTGCCCCA
CCCCAAGGTGTAACTGTATCCAAGAATGATGGAAACGGAACTGCAATTCT
AGTTAGTTGGCAGCCACCTCCAGAAGACACTCAAAATGGAATGGTCCAAG
AGTATAAGGTTTGGTGTCTGGGCAATGAAACTCGATACCACATCAACAAA
ACAGTGGATGGTTCCACCTTTTCCGTGGTCATTCCCTTTCTTGTTCCTGG
AATCCGATACAGTGTGGAAGTGGCAGCCAGCACTGGGGCTGGGTCTGGGG
TAAAG
Transmembrane Domain
AGATTTCAGATGTGGTGAAGCAGCCGGCCTTCATAGCAGGTATTGGAGCA
GCCTGTTGGATCATCCTCATGGTCTTCAGCATCTGGCTTTATCGACACCG
Cytoplasmic Motif #1
AATCTGAAGGATGGGCGTTTTGTCAATCCATCAGGGCAGCCTACTCCTTA
CGCCACCACTCAGCTCATCCAGTCAAACCTCAGCAACAACATGAACAATG
Cytoplasmic Motif #2
CCCAAGGTACCAAAACAGGGTGGCATGAACTGGGCAGACCTGCTTCCTCC
TCCCCCAGCACATCCTCCTCCACACAGCAATAGCGAAGAGTACAACATTT
Cytoplasmic Motif #3
CCAGCCAGGACATCTGCGCAGAGAAACCTACACAGATGATCTTCCACCAC
CTCCTGTGCCGCCACCTGCTATAAAGTCACCTACTGCCCAATCCAAGACA

TABLE 6
Hybridisation Probes for Human Roundabout 2

Immunoglobulin Domain #4
CAGATTGTTGCTCAAGGTCGAACAGTGACATTTCCCTGTGAAACTAAAGG
AAACCCACAGCCAGCTGTTTTTTGGCAGAAAGAAGGCAGCCAGAACCTAC
TTTTCCCAAACCAACCCCAGCAGCCCAACAGTAGATGCTCAGTGTCACCA
ACTGGAGACCTCACAATCACCAACATTCAACGTTCCGACGCGGGTTACTA
CATCTGCCAGGCTTTAACTGTGGCAGGAAGCATTTTAGCAAAAGCTCAAC
TGGAGGTTACTGATGTTTTGACA
Immunoglobulin Domain #5
GATAGACCTCCACCTATAATTCTACAAGGCCCAGCCAACCAAACGCTGGC
AGTGGATGGTACAGCGTTACTGAAATGTAAAGCCACTGGTGATCCTCTTC
CTGTAATTAGCTGGTTAAAGGAGGGATTTACTTTTCCGGGTAGAGATCCA
AGAGCAACAATTCAAGAGCAAGGCACACTGCAGATTAAGAATTTACGGAT
TTCTGATACTGGCACTTATACTTGTGTGGCTACAAGTTCAAGTGGAGAGG
CTTCCTGGAGTGCAGTGCTGGATGTGACAGAGTCT
Fibronectin Domain #1
GGAGCAACAATCAGTAAAAACTATGATTTAAGTGACCTGCCAGGGCCACC
ATCCAAACCGCAAGTCACTGATGTTACTAAGAACAGTGTCACCTTGTCCT
GGCAGCCAGGTACCCCTGGAACCCTTCCAGCAAGTGCATATATCATTGAG
GCTTTCAGCCAATCAGTGAGCAACAGCTGGCAGACCGTGGCAAACCATGT
AAAGACCACCCTCTATACTGTAAGAGGACTGCGGCCCAATACAATCTACT
TATTCATGGTCAGAGCGATCAACCCCAAGGTYTCAGTGACCCAAGT

TABLE 7
Primer Pairs for PCR of Human Roundabout 1 Domains

Immunoglobulin Domain #1
Forward: 5′ CCACCTCGCATTGTTGAACACCCTTCAGAC 3′
Reverse: 5′ ATGGCTACTTCCAGCGATGCATTGTGGCTC 3′
Immunoglobulin Domain #2
Forward: 5′ CTTCGGGATGACTTCAGACAAAACCCTTCG 3′
Reverse: 5′ TAAGACAGTCAGCTCGGCTACTTCACTCTC 3′
Immunoglobulin Domain #3
Forward: 5′ AGAGAGACCATCATTTGTGAAGAGACCCAG 3′
Reverse: 5′ AGGTTCTTGAACAGTCAGAGTAGCAGATGC 3′
Immunoglobulin Domain #4
Forward: 5′ CCACATTTTGTTGTGAAACCCCGTGACCAG 3′
Reverse: 5′ TGCAATCACATCTGTAACTTCCAAATATGC 3′
Immunoglobulin Domain #5
Forward: 5′ ATCGGCCTCCCCCAGTTATTCGACAAGGTC 3′
Reverse: 5′ CAAATTCTTGAACTTCAATGTAAGCACTCC 3′
Fibronectin Domain #1
Forward: 5′ GAGTTCCAGTTCAGCCTCCAAGACCTACTG 3′
Reverse: 5′ TCACTAATTCCATATGCATTAGCTGCCCTC 3′
Fibronectin Domain #2
Forward: 5′ CAAGCCAAATATCAGATCCAGTGAAAACAC 3′
Reverse: 5′ ATCTGCTCCTTGAAATTCATTAAAAAAAGG 3′
Fibronectin Domain #3
Forward: 5′ ATAGTGAAATCAAGTTTGCCAAAACCCTG 3′
Reverse: 5′ CTCTTTACCCCAGACCCAGCCCCAGTGCTG 3′
Transmembrane Domain
Forward: 5′ GGACCAAGTCAGCCTCGCTCAGCAGATTTC 3′
Reverse: 5′ ACTAGTAAGTCCGTTTCTCTTCTTGCGGTG 3′
Cytoplasmic Motif #1
Forward: 5′ CTGAAGGATGGGCGTTTTGTCAATCCATC 3′
Reverse: 5′ GTCCCAGTGGTTTCCAGTGCTTCTCGCCAG 3′
Cytoplasmic Motif #2
Forward: 5′ GGCACAAGAAAGGGGCAAGAACACCCAAGG 3′
Reverse: 5′ ATAGCTTTCATCTACAGAAATGTTGTACTC 3′
Cytoplasmic Motif #3
Forward: 5′ ACCAGACCAGCCAAGAAACTGAAACACCAG 3′
Reverse: 5′ GTACTTCCAGCTGTGTCTTGGATTGGGCAG 3′

TABLE 8
Human Roundabout 2 Primer Pairs

Immunoglobulin Domain #4
Forward: 5′ GTTGCTCAAGGTCGAACAGTGACATTTCCC 3′
Reverse: 5′ TGTCAAAACATCAGTAACCTCCAGTTGAGC 3′
Immunoglobulin Domain #5
Forward: 5′ GATAGACCTCCACCTATAATTCTACAAGGC 3′
Reverse: 5′ GACTCTGTCACATCCAGCACTGCACTCCAG 3′
Fibronectin Domain #1
Forward: 5′ CAATCAGTAAAAACTATGATTTAAGTG 3′
Reverse: 5′ TCGCTCTGACCATGAATAAGTAGATTG 3′

Such primers or probes are at least 12, preferably at least 24, more preferably at least 36 and most preferably at least 96 bases in length. Demonstrating specific hybridization generally requires stringent conditions, for example, hybridizing in a buffer comprising 30% formamide in 5×SSPE (0.18 M NaCl, 0.01 M NaPO₄, pH7.7, 0.001 M EDTA) buffer at a temperature of 42° C. and remaining bound when subject to washing at 42° C. with 0.2×SSPE; preferably hybridizing in a buffer comprising 50% formamide in 5×SSPE buffer at a temperature of 42° C. and remaining bound when subject to washing at 42° C. with 0.2×SSPE buffer at 42° C. Robo nucleic acids can also be distinguished using alignment algorithms, such as BLASTX (Altschul et al. (1990) Basic Local Alignment Search Tool, J Mol Biol 215, 403-410).

The subject nucleic acids are of synthetic/non-natural sequences and/or are isolated, i.e. unaccompanied by at least some of the material with which it is associated in its natural state, preferably constituting at least about 0.5%, preferably at least about 5% by weight of total nucleic acid present in a given fraction, and usually recombinant, meaning they comprise a non-natural sequence or a natural sequence joined to nucleotide(s) other than that which it is joined to on a natural chromosome. The subject recombinant nucleic acids comprising the nucleotide sequence of SEQ ID NO:1, 3, 5, 7, 9 or 11, or fragments thereof, contain such sequence or fragment at a terminus, immediately flanked by (i.e. contiguous with) a sequence other than that which it is joined to on a natural chromosome, or flanked by a native flanking region fewer than 10 kb, preferably fewer than 2 kb, more preferably fewer than 500 bp, which is at a terminus or is immediately flanked by a sequence other than that which it is joined to on a natural chromosome. While the nucleic acids are usually RNA or DNA, it is often advantageous to use nucleic acids comprising other bases or nucleotide analogs to provide modified stability, etc.

In a particular embodiment, expressed sequence tags EST;yu23d11, Accession #H77734 and EST;yq76e12, Accession #H52936, and deletion mutants thereof, are not within the scope of the present invention. In another embodiment, the subject Robo nucleic acids exclude the corresponding regions of the disclosed natural human Robo I nucleic acids, i.e. SEQ ID NO:7, nucleotides 500-651 and SEQ ID NO:7, nucleotides 3945-4455.

TABLE 10
Exemplary differences between H52936 and
corresponding human Robo I sequences.

(1) At position 86, there is a T instead of an A. The new codon
therefore reads TGA (Stop) instead of AGA (R).
(2) There is a missing G at position 286-7, causing a frameshift.
(3) There is an extra G at position 334, causing a frameshift.
(4) There is an extra T at position 344, causing a frameshift.
(5) There is an extra N at position 357, causing a frameshift.
(6) There is a T instead of a C at 362. The new codon reads
TTT (F) instead of TCT (S).
(7) There is an extra T at position 364, causing a frameshift.
(8) There is an extra N at position 370, causing a frameshift
and a changed amino acid (the codon TTN is ambiguous).
(9) There are two Ts at position 394 and 395 instead of a C,
causing a frameshift and amino acid changes.

TABLE 11
Exemplary differences between H52937 (reverse sequence)
and corresponding human Robo I sequences.

(1) There are multiple errors in the first 30 bases.
(2) At position 63, a G replaces an A. The new codon CGG
codes for R instead of CAG for Q.
(3) The EST ends by joining to part of the human glycophorin
B gene (353-442)

The subject nucleic acids find a wide variety of applications including use as translatable transcripts, hybridization probes, PCR primers, diagnostic nucleic acids, etc.; use in detecting the presence of Robo genes and gene transcripts and in detecting or amplifying nucleic acids encoding additional Robo homologs and structural analogs. In diagnosis, Robo hybridization probes find use in identifying wild-type and mutant Robo alleles in clinical and laboratory samples. Mutant alleles are used to generate allele-specific oligonucleotide (ASO) probes, for high-throughput clinical diagnoses. In therapy, therapeutic Robo nucleic acids are used to modulate cellular expression or intracellular concentration or availability of active Robo.

The invention provides efficient methods of identifying agents, compounds or lead compounds for agents active at the level of a Robo modulatable cellular function. Generally, these screening methods involve assaying for compounds which modulate Robo interaction with a natural Robo binding target. A wide variety of assays for binding agents are provided including labeled in vitro protein-protein binding assays, immunoassays, cell based assays, etc. The methods are amenable to automated, cost-effective high throughput screening of chemical libraries for lead compounds. Identified reagents find use in the pharmaceutical industries for animal and human trials; for example, the reagents may be derivatized and rescreened in in vitro and in vivo assays to optimize activity and minimize toxicity for pharmaceutical development.

Cell and animal based neural guidance/repulsion assays are described in detail in the experimental section below. In vitro binding assays employ a mixture of components including a Robo polypeptide, which may be part of a fusion product with another peptide or polypeptide, e.g. a tag for detection or anchoring, etc. The assay mixtures comprise a natural intracellular Robo binding target. While native full-length binding targets may be used, it is frequently preferred to use portions (e.g. peptides) thereof so long as the portion provides binding affinity and avidity to the subject Robo polypeptide conveniently measurable in the assay. The assay mixture also comprises a candidate pharmacological agent. Candidate agents encompass numerous chemical classes, though typically they are organic compounds; preferably small organic compounds and are obtained from a wide variety of sources including libraries of synthetic or natural compounds. A variety of other reagents may also be included in the mixture. These include reagents like salts, buffers, neutral proteins, e.g. albumin, detergents, protease inhibitors, nuclease inhibitors, antimicrobial agents, etc. may be used.

The resultant mixture is incubated under conditions whereby, but for the presence of the candidate pharmacological agent, the Robo polypeptide specifically binds the cellular binding target, portion or analog with a reference binding affinity. The mixture components can be added in any order that provides for the requisite bindings and incubations may be performed at any temperature which facilitates optimal binding. Incubation periods are likewise selected for optimal binding but also minimized to facilitate rapid, high-throughput screening.

After incubation, the agent-biased binding between the Robo polypeptide and one or more binding targets is detected by any convenient way. Where at least one of the Robo or binding target polypeptide comprises a label, the label may provide for direct detection as radioactivity, luminescence, optical or electron density, etc. or indirect detection such as an epitope tag, etc. A variety of methods may be used to detect the label depending on the nature of the label and other assay components, e.g. through optical or electron density, radiative emissions, nonradiative energy transfers, etc. or indirectly detected with antibody conjugates, etc.

A difference in the binding affinity of the Robo polypeptide to the target in the absence of the agent as compared with the binding affinity in the presence of the agent indicates that the agent modulates the binding of the Robo polypeptide to the Robo binding target. For example, in the cell-based assay also described below, a difference in Robo-dependent modulation of axon outgrowth or orientation in the presence and absence of an agent indicates the agent modulates Robo function. A difference, as used herein, is statistically significant and preferably represents at least a 50%, more preferably at least a 90% difference.

The following experimental section and examples are offered by way of illustration and not by way of limitation.

### EXPERIMENTAL

Cloning of the roundabout Gene. The robo¹ allele was mapped to the plexus-brown interval on the right arm of the second chromosome by recombination mapping; the numbers of recombinants suggested a map position very close to plexus at 58F/59A. One deficiency [Df(2R)P, which deletes 58E3/F1 through 60D14/E2] fails to complement robo mutations, two other deficiencies [Df(2R)59AB and Df(2R)59AD, which delete 59A1/3 through 59B1/2 and 59A1/3 through 59D1/4 respectively] do complement robo, and a duplication [Dp(2;Y)bw⁺Y, which duplicates 58F1/59A2 through 60E3/F1] rescues robo mutations. This mapping places robo in the 58F/59A region.

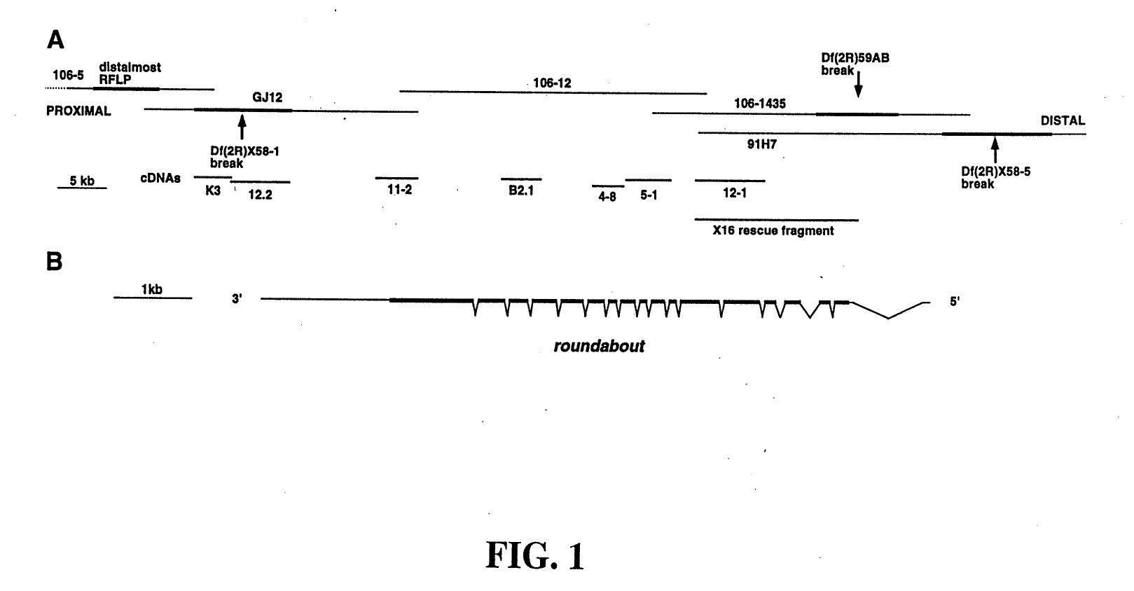

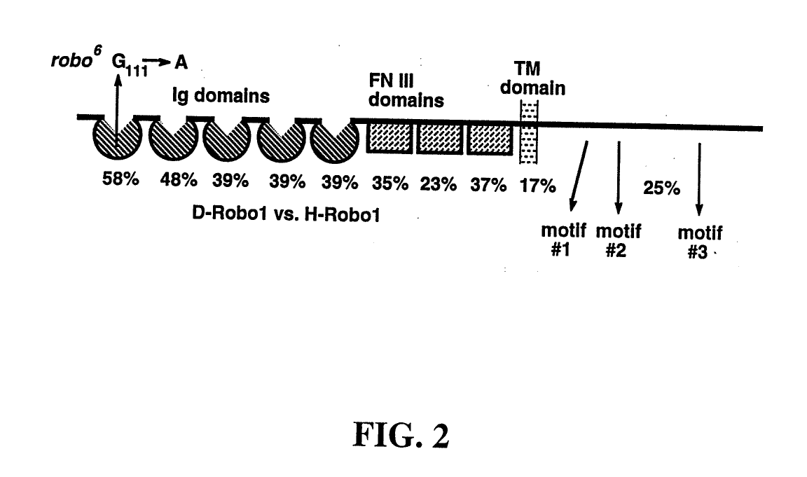

We initiated chromosomal walks from P1 clones mapped to the region, beginning from the distal side using clone DS02204 and from the proximal side using clone DS05609. We used cosmid clones (Tamkun et al., 1992) to complete a walk of ˜150 kb. We then looked for RFLPs in the recombinants between the multiple marked chromosome and the robo mutant chromosome. A 6.8 kb EcoRI fragment from cosmid 106-5 identified a HindII RFLP on the mapping chromosome that was present on a single robo mutant recombinant line. This fragment identified a proximal limit for the location of robo. Further deficiencies in this region were then tested (Kerrebrock et al., 1995). Of these deficiencies, Df(2R)X58-5 and Df(2R)X58-12 remove robo while Df(2R)X58-1 does not. Df(2R)X58-12 fails to complement Df(2R)59AB yet complements Df(2R)59AD indicating that Df(2R)59AB extends further proximal; this proximal endpoint provides a distal limit for the location of robo. Probes from the walk were used to identify the breakpoints of these deficiencies (FIG. 1A). Df(2R)X58-1 breaks in a 9.6 kb EcoRI/BamHI fragment within cosmid GJ12, whereas Df(2R) 59AB breaks in a 8 kb BamHI/EcoRI fragment within cosmid 106-1435. This reduces the location of robo to a 75 kb region bounded by these restriction fragments. Hybridization of 0-16 hr poly-A⁺ embryonic Northern blots with cosmids GJ12, 106-12, and 106-1435 revealed at least five transcripts. Reverse Northern mapping identified the regions containing these transcripts (FIG. 1A). These regions were used as probes to isolate cDNAs. Seven different cDNAs were isolated and analyzed by in situ hybridization. The expression pattern of five of these transcripts allowed us to tentatively discount them as encoding for robo since they were not expressed in the embryonic CNS at the appropriate stage. Of the two cDNAs remaining, 12-1 appeared by its size and expression the most likely candidate for robo. A 16 kb XbaI fragment including the 12-1 transcript and a region 5′ to the transcript is capable of rescuing the robo mutant.

roundabout Encodes a Member of the Immunoglobulin Superfamily. We recovered and sequenced overlapping cDNA clones corresponding to the 12-1 transcription unit. A single long open reading frame (ORF) that encodes 1395 amino acids was identified (D1 in Table 1). Conceptual translation of the ORF reveals the Robo protein to be a member of the Ig superfamily; Robo's ectodomain contains five immunoglobulin (Ig)-like repeats followed by three fibronectin (Fn) type-III repeats. The predicted ORF also contains a transmembrane domain and a large 457 amino acid (a.a.) cytoplasmic domain. Hydropathy analysis of the Robo sequence indicates a single membrane spanning domain of 25 a.a. (Kyte and Doolittle, 1982) plus a signal sequence with a predicted cleavage site between G51 and Q52 (Nielsen et al 1997).

We identify the 12-1 transcript as encoding robo based on several criteria. First, the embryonic robo phenotype can be rescued by the 16 kb XbaI genomic fragment containing this cDNA; no other transcripts are contained in this 16 kb XbaI fragment. Second, we identified a CfoI RFLP associated with the allele robo⁶. This polymorphism is due to a change of nucleotide 332 of the ORF from G to A, which results in a change of Gly₁₁₁ to Asp. Gly111 is in the first Ig domain (FIG. 2), and is conserved in all Robo homologues identified. The change is specific to the allele robo⁶ and is not seen in the parental chromosome or in any of the other seven alleles, all of which were generated from the same parental genotype. Third, the production of antibodies (below) which recognize the Robo protein reveals that the alleles robo¹, robo², robo³, robo⁴ and robo⁵ do not produce Robo protein (Table 12).

TABLE 12
robo Mutant Alleles

| Allele | Synonym | Class |
|---|---|---|
| robo¹ | GA285 | Protein null |
| robo² | GA1112 | Protein null |
| robo³ | Z14 | Protein null |
| robo⁴ | Z570 | Protein null |
| robo⁵ | Z1772 | Protein null |
| robo⁶ | Z1757 | Protein positive; Gly₁₁₁ to Asp |
| robo⁷ | Z2130 | Reduced protein levels |
| robo⁸ | Z3127 | Protein positive |

All alleles were generated by EMS mutagenesis of FasIII null chromosomes. Each of these alleles appear to represent a complete, or near complete, loss-of-function phenotype for robo, since the mutant phenotype observed when these alleles are placed over a chromosome deficient for the robo locus [Df(2R) X58-5] is indistinguishable from the homozygous allele.

Finally, transgenic neural expression of robo rescues the midline crossing phenotype of robo mutants (see below).

Developmental Northern blot analysis using both cDNA and genomic probes suggests that robo is encoded by a single transcript of ˜7500 bp. We sequenced genomic DNA and identified 17 introns within the sequence of which 14 are only 50-75 by in length plus three introns of 843 bp, 236 bp, and 110 by (FIG. 1B). The precise start point of the transcript has not been determined.

A Family of Evolutionarily Conserved Robo-like Proteins. The presence of five Ig and three Fn domains, a transmembrane domain, and a long (452 a.a.) cytoplasmic region indicates that Robo may be a receptor and signaling molecule. The netrin receptor DCC/Frazzled/UNC-40 has a related domain structure, with 6 Ig and 4 Fn domains and a similarly long cytoplasmic region (Keino-Masu et al., 1996; Chan et al., 1996; Kolodziej et al., 1996). The only currently known protein with a “5+3” organization is CDO (Kang et al., 1997). However, CDO is only distantly related to Robo (15-33% a.a. identity between corresponding Ig and FN domains).

We identified other “5+3” proteins in vertebrates whose amino acid identity exceeds that of CDO and represent Robo homologues. A human expressed sequence tag (EST; yu23d11, Accession #H77734) shows high homology to the second Ig domain of robo and was used to probe a human fetal brain cDNA library (Stratagene). The clones recovered correspond to a human gene with five Ig and three Fn domains (FIG. 2). Exemplary functional Robo domains are listed in Tables 13-17 (the corresponding encoding nucleic acids are readily discernable from the corresponding nucleic acid sequences of Sequence Listing).

TABLE 13
Exemplary domains of human Robo 1,
by amino acid sequence positions

| | |
|---|---|
| Signal sequence: | 6-21 |
| First Immunoglobulin domain: | 68-167 |
| Second Immunoglobulin domain: | 168-258 |
| Third Immunoglobulin domain: | 259-350 |
| Fourth Immunoglobulin domain: | 351-450 |
| Fifth Immunoglobulin domain: | 451-546 |
| First Fibronectin domain: | 547-644 |
| Second Fibronectin domain: | 645-761 |
| Third Fibronectin domain: | 762-862 |
| Transmembrane domain: | 896-917 |
| Cytoplasmic motif #1: | 1070-1079 |
| Cytoplasmic motif #2: | 1181-1195 |
| Cytoplasmic motif #3: | 1481-1488 |

TABLE 14
Exemplary domains of human Robo II,
by amino acid sequence positions

| | |
|---|---|
| Fourth Immunoglobulin domain: | 1-91 |
| Fifth Immunoglobulin domain: | 92-185 |
| First Fibronectin domain: | 186-282 |

TABLE 15
Exemplary domains of drosophila Robo
1, by amino acid sequence positions

| | |
|---|---|
| Signal sequence: | 30-46 |
| First Immunoglobulin domain: | 56-152 |
| Second Immunoglobulin domain: | 153-251 |
| Third Immunoglobulin domain: | 252-344 |
| Fourth Immunoglobulin domain: | 345-440 |
| Fifth Immunoglobulin domain: | 441-535 |
| First Fibronectin domain: | 536-635 |
| Second Fibronectin domain: | 636-753 |
| Third Fibronectin domain: | 754-854 |
| Transmembrane domain: | 915-938 |
| Cytoplasmic motif #1: | 1037-1046 |
| Cytoplasmic motif #2: | 1098-1119 |
| Cytoplasmic motif #3: | 1262-1269 |

TABLE 16
Exemplary domains of drosophila Robo
II, by amino acid sequence positions

| | |
|---|---|
| Immunoglobulin domain #1: | 4-99 |
| Immunoglobulin domain #2: | 100-192 |
| Immunoglobulin domain #3: | 193-296 |
| Immunoglobulin domain #4: | 297-396 |
| Immunoglobulin domain #5: | 397-494 |
| Fibronectin domain #1: | 495-595 |
| Fibronectin domain #2: | 596-770 |
| Fibronectin domain #3: | 771-877 |
| Transmembrane domain: | 906-929 |
| Conserved cytoplasmic motif #1: | 1075-1084 |

TABLE 17
Exemplary domains of C. elegans Robo
1, by amino acid sequence positions

| | |
|---|---|
| First Immunoglobulin domain: | 30-129 |
| Second Immunoglobulin domain: | 130-223 |
| Third Immunoglobulin domain: | 224-315 |
| Fourth Immunoglobulin domain: | 316-453 |
| Fifth Immunoglobulin domain: | 454-543 |
| First Fibronectin domain: | 544-643 |
| Second Fibronectin domain: | 644-766 |
| Third Fibronectin domain: | 767-865 |
| Transmembrane domain: | 900-922 |
| Cytoplasmic motif #1: | 1036-1045 |
| Cytoplasmic motif #2: | 1153-1163 |
| Cytoplasmic motif #3: | 1065-1074 |

The homology is particularly high in the first two Ig domains (58% and 48% a.a. identity respectively, compared to 26% and 30% for the same two Ig domains between D-Robo1 and CDO) and together with the overall identity throughout the extracellular region and the presence of three conserved cytoplasmic motifs has led us to designate this as the human roundabout 1 gene (H-robo1). Database searching reveals a nucleotide sequence corresponding to H-robo1 in the database, DUTT1, which differs in the signal sequence suggesting alternative splicing, a 9 by insertion and seven single base pair changes. Five ESTs (see Experimental Procedures) show high sequence similarity to the cytoplasmic domain of H-robo1. Sequencing of cDNAs isolated using one of these ESTs as a probe confirmed a second human roundabout gene (H-robo2).

Degenerate PCR primers based on conserved sequences between H-robo1 and D-robo1 were used to isolate a PCR fragment from a rat embryonic E13 brain cDNA library. The fragment was used to probe an E13 spinal cord cDNA library, resulting in the isolation of a full length Rat robo gene (R-robo1). The predicted protein shows high sequence identitiy (>95%) with H-robo1 over the entire length. The 5′ sequences of different R-robo1 cDNA clones indicates that this gene is alternatively spliced in a similar fashion to H-robo1/DUTTI. We used a similar approach to isolate cDNA clones for R-robo2, which is highly homologous to H-robo2.

The mouse EST vi92e02 is highly homologous to the cytoplasmic portion of H-robo1. The C. elegans Sax-3 gene is also a robo homologue (Table 1; Zallen et al., 1997). A second Drosophila robo gene (D-robo2) is also predicted from analysis of genomic sequence in the public database. Taken together these data indicate that Robo is the founding member of a new subfamily of Ig superfamily proteins with at least one member in nematode, two in Drosophila, two in rat, and two in human.

The alignment of the Robo family proteins reveals that the first and second Ig domains are the most highly conserved portion of the extracellular domain. The cytoplasmic domains are highly divergent except for the presence of three highly conserved motifs (Table 18).

TABLE 18

Conserved Cytoplasmic Motifs: Amino acid alignments of the
three conserved cytoplasmic motifs are shown below the
structure; in C. elegans robo, motifs #2 and #3 have
been switched to provide a better alignment.

Conserved Cytoplasmic Motif #1

| | |
|---|---|
| PDNPTPYATTMIIGTSS | 1050 Drosophila roundabout-I |
| SGQPTPYATTQLIQSNL | 1083 Human roundabout-I |
| NASPAPYATSSILSPHQ | 1088 Drosophila roundabout-II |
| HDDPSPYATTTLVLSNQ | 1049 C. elegans roundabout |
| PtPYATT.hh.... | Consensus (where h is I, L or V) |

Conserved Cytoplasmic Motif #2

| | |
|---|---|
| INWSE.FLPPPPEHPPPSSTYG.Y | 1119 Drosophila roundabout-I |
| MNWAD.LLPPPPAHPPPHSNSEEY | 1202 Human roundabout-I |
| STWANVPLPPPPVQPLPGTELEHY | 31 Human roundabout-II |
| KTLMD.FIPPPPSNPPPP.GGHVY | 1168 C. elegans roundabout-I |
| nW...hhPPPP. PPP.s....Y | Consensus (where h is hydrophobic) |

Conserved Cytoplasmic Motif #3

| | |
|---|---|
| PSPMQPPPPVPVPEGW.Y | 1273 Drosophila roundabout-I |
| YTDDLPPPPVPPPAIKSP | 1493 Human roundabout-I |
| YADDLPPPPVPPPAIKSP | 90 Mouse roundabout-I |
| RAPAMPTNPVPPEPPARY | 1077 C. elegans roundabout |
| .....PPPPVPPP.... | Consensus |

The consensus for the first motif is PtPYATTxhh, where x is any amino acid and h is I, L, or V. The presence of a tyrosine in the center of the motif indicates a site for phosphorylation. The other two motifs consist of runs of prolines separated by one or two amino acids and are reminiscent of binding sites for SH3 domains. In particular, the LPPP sequence in motif #2 provides a good binding site for the Drosophila Enabled protein or its mammalian homologue Mena (Niebuhr et al., 1997). All three of these conserved sites can function as binding sites for domains (e.g. SH3 domains) of linker/adapter proteins functioning in Robo-mediated signal transduction.

Robo is Regionally Expressed on Longitudinal Axons in the Drosophila Embryo. In order to determine the role that robo might play in regulating axon crossing behavior, we examined the robo expression pattern in the embryonic CNS. The in situ hybridization pattern of robo mRNA in Drosophila shows it to have elevated and widespread expression in the CNS. We raised a monoclonal antibody (MAb 13C9) against part of the extracellular portion (amino acids 404-725) of the protein to visualize Robo expression. Robo is first seen in the embryo weakly expressed in lateral stripes during germband extension. At the onset of germband retraction, Robo expression is observed in the neuroectoderm. By the end of stage 12, as the growth cones first extend, Robo is seen on growth cones which project ipsilaterally, including pCC, aCC, MP1, dMP2, and vMP2. Strikingly, little or no Robo expression is observed on commissural growth cones as they extend towards and across the midline. However, as these growth cones turn to project longitudinally, their level of Robo expression dramatically increases. Robo is expressed at high levels on all longitudinally-projecting growth cones and axons. In contrast, Robo is expressed at nearly undetectable levels on commissural axons. This is striking since ˜90% of axons in the longitudinal tracts also have axon segments crossing in one of the commissures. Thus, Robo expression is regionally restricted. Robo expression is also seen at a low level throughout the epidermis and at a higher level at muscle attachment sites. In stage 16-17 embryos, faint Robo staining can be seen in the commissures but at levels much lower than observed in the longitudinal tracts.

Immunoelectron Microscopy of Robo. We used immunoelectron microscopy to examine Robo localization at higher resolution. In stage 13 embryos, Robo is expressed at higher levels on growth cones and filopodia in the longitudinal tracts than on the longitudinal axons themselves. This localization is consistent with the model that Robo functions as a guidance receptor. The increased sensitivity of immunoelectron microscopy reveals the presence of very low levels of Robo protein on the surface of commissural axons. In addition, Robo-positive vesicles can be seen inside the commissural axons, possibly representing transport of Robo to the growth cone. Finally, by reconstructing the path of single axons by use of serial sections, we confirm that Robo expression is greatly up-regulated after individual axons turn from the commissure into a longitudinal tract. The expression of Robo on non-crossing and post-crossing axons and its higher level of expression on growth cones and its filopodia, provide a model where Robo functions as an axon guidance receptor for a repulsive midline cue.

Transgenic Expression of Robo. We hypothesized that if Robo is indeed a growth cone receptor for a midline repellent, then pan-neural expression of Robo protein during the early stages of axon outgrowth might lead to a robo gain-of-function phenotype similar to the comm loss-of-function and opposite of the robo loss-of-function. To test this hypothesis, we cloned a robo cDNA containing the complete ORF but lacking most of its untranslated regions (UTRs) downstream of the UAS promoter in the pUAST vector and generated transgenic flies for use in the GAL4 system (Brand and Perrimon, 1993). Expression of robo in all neurons was achieved by crossing the UAS-robo flies to either the elav-GAL4 or scabrous-GAL4 lines.

Surprisingly, pan-neural expression of robo mRNA did not produce a strong axon scaffold phenotype as assayed with MAb BP102. Staining with anti-Fas II (MAb 1D4) revealed subtle fasciculation defects, but overall the axon scaffold looked quite normal. An insight into why we failed to observe a stronger robo ectopic expression phenotype was provided by staining these embryos with the anti-Robo MAb. Interestingly, the Robo protein, although expressed at higher levels than in wild type, remains restricted as in wild type, i.e., high levels of expression on the longitudinal portions of axons and very low levels on the commissures. This result indicates that there must be strong regulation of Robo expression, probably post-translational, that assures its localization to longitudinal axon segments. Such a mechanism could operate by the regulation of protein translation, transport, insertion, internalization and/or stability.

We used these transgenic flies to rescue robo mutants. Expression of robo by the elav-GAL4 line in both robo³ and robo⁵ homozygotes rescued the midline crossing of Fas II positive axons including pCC and other identified neurons.

Robo Appears to Function in a Cell Autonomous Fashion. To test whether Robo can function in a cell autonomous fashion, we used the UAS-robo transgene with the ftz_{ng}-GAL4 line (Lin et al., 1994). The ftz_{ng}-GAL4 line expresses in a subset of CNS neurons, including many of the earliest neurons to be affected by the robo mutation such as pCC, vMP2, dMP2, and MPI. Expression of robo by the ftz_{ng}-GAL4 line is sufficient to rescue these identified neurons in the robo mutant: pCC, which in robo mutants heads towards and crosses the midline, in these rescued embryos now projects ipsilaterally and does not cross the midline. When the same embryos were stained with the anti-robo MAb 13C9, we observed that all Robo-positive axons did not cross the midline. The ftz_{ng}-GAL4 line drives expression in many of the axons in the pCC pathway (Lin et al., 1994), a medial longitudinal fascicle. In robo mutants, this axon fascicle freely crosses and circles the midline, joining with its contralateral pathway. When rescued by the ftz_{ng}-GAL4 line driving UAS-robo, this pathway now largely remains on its own side of the midline, even though occasionally a few axons cross the midline. These experiments support the notion that Robo can function in a cell autonomous fashion.

Expression of Mammalian robo1 in the Rat Spinal Cord. The isolation of several vertebrate Robo homologues suggests that Robo may play a similar role in orchestrating midline crossing in the vertebrate nervous system as it does in Drosophila. In the vertebrate spinal cord, the ventral midline is comprised of a unique group of cells called the floor plate (for review, Colamarino and Tessier-Lavigne, 1995). As in the Drosophila nervous system, the vertebrate spinal cord contains both crossing and non-crossing axons. Spinal commissural neurons are born in the dorsal half of the spinal cord; commissural axons project to and cross the floor plate before turning longitudinally in a rostral direction. In contrast, the axons of two other classes of neurons, dorsal association neurons and ventral motor neurons, do not cross the floor plate (Altman and Bayer, 1984).

To address the possibility that Robo may play a role in organizing the projections of these spinal neurons, we examined the expression of rat robo1 by RNA in situ hybridization. A rat robo1 riboprobe spanning the first three Ig domains was hybridized to transverse sections of E13 rat spinal cord. At E13, when many commissural axons will have already extended across the floor plate (Altman and Bayer, 1984), rat robo1 is expressed at high levels in the dorsal spinal cord, in a pattern corresponding to the cell bodies of commissural neurons. Rat robo1 is also expressed at lower levels in a subpopulation of ventral cells in the region of the developing motor column. Interestingly, this expression pattern is similar to and overlaps partly with the mRNA encoding DCC, another Ig superfamily member which is also expressed on commissural and motor neurons and encodes a receptor for Netrin-1 (Keino-Masu et al, 1996). Rat robo1 is not, however, expressed in the either the floor plate or the roof plate of the spinal cord or in the dorsal root ganglia. This is in contrast to rat cdo, which is strongly expressed in the roof plate (KB, MT-L, and R. Krauss. In the periphery, rat robo1 is also found to be expressed in the myotome and developing limb, in a pattern reminiscent of c-met (Ebens et al, 1996), indicating that rat robo1 may also be expressed by migrating muscle precursor cells. Therefore, like its Drosophila homologue, rat robo1 RNA is expressed by both crossing and non-crossing populations of axons, indicating that it encodes the functional equivalent of D-Robo1.

Genetic Stocks. All eight independent robo alleles were isolated on chromosomes deficient for Fasciclin III as described in Seeger et al., 1993. Subsequent use of a duplication that includes FasIII, and recombination of the robo chromosomes, indicates that the robo phenotype is independent of the absence of FasIII. Deficiencies were obtained from the Drosophila stock center at Bloomington, Ind.

Cloning and Molecular Analysis of the robo Genes. Start points for a molecular walk to robo were obtained from the Berkeley and Crete Drosophila Genome Projects. Chromosomal walking was performed using standard techniques to isolate cosmids from the Tamkun library (Tamkun et al., 1992). cDNAs were isolated from the Zinn 9-12 hour Drosophila embryo gtll library (Zinn et al., 1988), and from a human fetal brain library (Stratagene). Northern blot of poly-A⁺RNA and reverse Northern blots were hybridized using sensitive Church conditions.

Sequencing of the cDNAs and genomic subclones was performed by the dideoxynucleotide chain termination method using Sequenase (USB) following the manufacturer's protocol and with the AutoRead kit or AutoCycle kit (Pharmacia) or by ³³P cycle sequencing. Reactions were analyzed on a Pharmacia LKB or ABI automated laser fluorescent DNA sequencers respectively. The cDNAs were sequenced completely on both strands. Sequence contigs were compiled using Lasergene, Intelligenetics, and AssemblyLIGN software (Kodak Eastman). Database searches were performed using BLAST (Altschuel et al., 1990).

A full length D-robo1 cDNA was generated by ligating two partial cDNAs at an internal HpaI site and subcloning into the EcoRI site of pBluescript.SK+. A full length H-robo1 cDNA was synthesized by ligating an XbaI-SalI fragment from a cDNA and a PCR product coding for the carboxy-terminal 222 amino acids at a SalI site. The PCR product has an EcoRI site introduced at the stop codon. The ligation product was cloned into pBluescript.SK+ digested with XbaI and EcoRI.

To clone the rat robo1 cDNA, degenerate oligonucleotide primers designed against sequences conserved between the 5′ ends of D-Robo1 and H-Robo1 were used to amplify a 500 by fragment from an E13 rat brain cDNA by PCR. This fragment was used to screen an E13 spinal cord library at high stringency, resulting in the isolation of a 4.2 kb cDNA clone comprising all but the last 700 nucleotides. Subsequent screenings of the library with non-overlapping probes from this cDNA led to the isolation of 4 partial and 7 full length clones. To clone the rat robo2 cDNA, we screened the same library with a fragment of the H-robo2 cDNA.

Expressed Sequence Tag and Genomic Sequences. The ESTs yu23d11 (#H77734), zr54g12 (#AA236414) and yq76e12 (#H52936, #H52937) code for portions of H-Robo1. The EST yq7e12 is aberrantly spliced to part of the human glycophorinB gene. Five ESTs yn50a07, yg02b06, ygl7b06, yn13a04 and yml7g11 code for part of H-robo2. The Drosophila P1 clone DS00329 encodes the genomic sequence of D-robo2. Sequences 1825710 and 1825711 (both: #U88183; locus ZK377) code for the predicted sequence of C. elegans robo. The EST vi62e02 (#AA499193) codes for mouse robo1 .

Identification of Molecular Defects In robo Alleles. Southern blots of robo alleles and their parental chromosomes were hybridized with fragments from the genomic cosmid clone 106-1435 or partial cDNA clones to identify restriction fragment length polymorphisms affecting the robo transcription unit. DNA was obtained from homozygous mutant embryos. 35 cycles of the PCR was subsequently performed on the DNA obtained from half an embryo. Primers specific for the region flanking the CfoI polymorphism used were: ROBO6 (5′-GCATTGGGTCATCTGTAGAG-3′) and ROBO23 (5′-AGCTATCTGGAGGGAGGCAT-3′). The PCR products were purified on a Pharmacia H300 spin column and sequenced directly.

Transformation of Drosophila, robo Rescue, and Overexpression. The 16 kb XbaI fragment from cosmid 106-1435 was cloned into the Drosophila transformation vector pCaSpeR3. Transformant lines were generated and mapped by standard procedures. Four independent lines were shown to rescue robo^{1,3,5} alleles as judged by MAb 1D4 staining.

PCR amplification of the D-robo ORF using the primers (5′-GAGTGGTGAATTCAACAGCACCAAAACCACAAAATGCATCCC-3′) and (5′-CGGGGAGTCTAGAACACTTCATCCTTAGGTG-3′) produced a PCR product with an altered ribosome binding site that more closely matches the Drosophila consensus (Cavener, 1987), and has only 21 bp of 5′ UTR and no 3′ UTR sequences. The PCR product was digested with EcoRI and XbaI and cloned into pBluescript (Stratagene) and subsequently, pUAST (Brand and Perrimon 1993). Transformant lines were crossed to elav-GAL4 and sca-GAL4 lines which express GAL4 in all neurons, or ftzng-GAL4 which expresses in a subset of CNS neurons (Lin et al, 1994). Embryos were assayed by staining with MAbs BP102, 1D4 and 13C9. For ectopic expression in the robo mutant background, the stocks robo³ and robo⁵ (both protein nulls) were used. Crosses utilized the stocks w; robo/CyO; UAS-robo and w; robo/CyO; elav-GAL4. Due to the difficulty of maintaining a balanced stock, robo/+; ftz-ngGAL4/+ males were generated as required.

Generation of Fusion Proteins and Antibodies. A six histidine tagged fusion protein was constructed by cloning amino acids 404-725 of the D-robo protein into the PstI site of the pQE31 vector (Qiagen). Fusion proteins were purified under denaturing conditions and subsequently dialyzed against PBS. Immunization of mice and MAb production followed standard protocols (Patel, 1994).

RNA Localization and Protein Immunocytochemistry. Digoxigenin labeled antisense robo transcripts were generated from a subclone of a robo cDNA in Bluescript. In-situ tissue hybridization was performed as described in Tear et al., 1996. Immunocytochemistry was performed as described by Patel, 1994. MAb 1D4 was used at a dilution of 1:5 and BP102 at 1:10. For anti-robo staining, MAb 13C9 was diluted 1:10 in PBS with 0.1% Tween-20, and the embryos were fixed and cracked so as to minimize exposure to methanol. The presence of triton and storage of embryos in methanol were both found to destroy the activity of MAb 13C9.

In situ hybridization of rat spinal cords was carried out essentially as described in Fan and Tessier-Lavigne, 1994. E13 embryos were fixed in 4% paraformaldehyde, processed, embedded in OCT, and sectioned to 10 m. A 1.0 kb ³⁵S antisense rRobo riboprobe spanning the first three immunoglobulin domains was used for hybridization. An additional non-overlapping probe was also used with identical results. DCC transcripts were detected as described in Keino-Masu et al., 1996. Immunohistochemistry against TAG-1 was carried out on 10 m transverse spinal cord sections using 4D7 monoclonal antibody (Dodd et al, 1988).

Electron Microscopy. Canton S embryos were hand devitellinized, opened dorsally to remove the gut, and prepared for immunoelectron microscopy according to the procedures described previously (Lin et al., 1994), with the following modifications. The fixed embryos were incubated sequentially with MAb 13C9 (1:1) for 1-2 hours, biotinylated goat anti-mouse secondary antibody (1:250) for 1.5 hours, and then streptavidin-conjugated HRP (1:200) for 1.5 hours. Hydrogen peroxide (0.01%) was used instead of glucose oxidase for the HRP-DAB reaction.

### REFERENCES

Altman, J. and Bayer, S. A. (1984) Adv. Anat. Embryol. Cell Biol. 85, 1-164.
Altschul, S. F., et al. (1990) J. Mol. Biol. 215, 403-410.
Bastiani, M. J., et al. (1987) Cell 48, 745-755.
Brand, A. H., and Perrimon, N. (1993) Development 118, 401-415.
Cavener, D. (1987) Nucl. Acids Res. 15, 1353-1361.
Chan, S. et al. (1996) Cell 87, 187-195.
Dodd, J., et al. (1988) Neuron 1, 105-116.
Ebens, A., et al. (1996) Neuron 17, 1157-1172.
Elkins, T., et al. (1990) Cell 60, 565-575.
Fan, C. M. and Tessier-Lavigne, M. (1994) Cell 79, 1175-1186.
Gertler, F. B., et al. (1995) Genes Develop. 9, 521-533.
Harris, R., Sabatelli, L. M., and Seeger, M. A. (1996) Neuron 17, 217-228.
Hedgecock, E. M., Culotti, J. G., and Hall, D. H. (1990) Neuron 4, 61-85.
Kang, J-S., et al. (1997) J. Cell Biol. 138, 203-213.
Keino-Masu, K., et al. (1996) Cell 87, 175-185.
Kennedy, T. E., et al. (1994) Cell 78, 425-435.
Kerrebrock, A. W., et al. (1995) Cell 83, 247-256.
Kidd, T., Russell, C., Goodman, C. S., and Tear, G. (1997). Dosage sensitive and complementary functions of Roundabout and Commissureless control axon crossing of the CNS midline. Neuron, in review.
Kolodziej, P. A., et al. (1996) Cell 87, 197-204.
Kyte, J., and Doolittle, R. F. (1982) J. Mol. Biol. 157, 105-132.
Lin, D. M., et al. (1994) Neuron 13, 1055-1069.
Mitchell, K. J., et al. (1996) Neuron 17, 203-215.
Myers, P. Z., and Bastiani, M. J. (1993) Journal of Neuroscience 13, 127-143.
Niebuhr, K., et al. (1997) EMBO J. 16, 5433-5444.
Nielsen, H., et al. (1997) Protein Engineering 10, 1-6.
Patel, N. H. (1994) In “Methods in Cell Biology, Vol 44. Drosophila melanogaster: Practical Uses in Cell Biology” (L. S. B. Goldstein and E. Fyrberg, eds) Academic Press, New York.
Seeger, M., Tear, G., Ferres-Marco, D., and Goodman C. S. (1993) Neuron 10, 409-426.
Serafini, T., et al. (1994) Cell 78, 409-424.
Stoeckli, E. T., and Landmesser, L. T. (1995) Neuron 14, 1165-1179.
Stoeckli, et al. (1997) Neuron 18, 209-221.
Tamkun, J. W., et al. (1992) Cell 68, 561-572.
Tear, G., et al. (1993) Perspectives on Developmental Neurobiology 1, 183-194.
Tear G., et al. (1996) Neuron 16, 501-514.
Tessier-Lavigne, M., and Goodman, C. S. (1996) Science 274, 1123-1133.
Wadsworth, W. G., Bhatt, H., and Hedgecock, E. M. (1996) Neuron 16, 35-46.
Zallen, J., Yi, A., and Bargmann, C. (1997). The conserved immunoglobulin superfamily member SAX-3/Robo directs multiple aspects of axon guidance in C. elegans. Cell, in review.
Zinn, K., McAllister, L., and Goodman, C. S. (1988)Cell 53, 577-587.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. An isolated antibody that specifically binds to a polypeptide of SEQ ID NO:8.

2. The antibody of claim 1, wherein the antibody is a polyclonal antibody.

3. The antibody of claim 1, wherein the antibody is a monoclonal antibody.

4. The antibody of claim 1, wherein the antibody binds to an extracellular domain of the polypeptide.

5. The antibody of claim 1, wherein the antibody binds to a domain selected from the group consisting of:
(a) residues 68-167 of SEQ ID NO:8;
(b) residues 168-258 of SEQ ID NO:8;
(c) residues 259-350 of SEQ ID NO:8;
(d) residues 351-450 of SEQ ID NO:8;
(e) residues 451-546 of SEQ ID NO:8;
(f) residues 547-644 of SEQ ID NO:8;
(g) residues 645-761 of SEQ ID NO:8; and
(h) residues 762-862 of SEQ ID NO:8.

6. The antibody of claim 1, wherein the antibody binds to a fragment of SEQ ID NO:8 selected from the group consisting of residues 18-28, 31-40, 45-65, 106-116, 137-145, 174-184, 214-230, 274-286, 314-324, 399-412, 496-507, 548-565, 599-611, 660-671, 717-730, 780-791, 835-847, 877-891, 930-942, 981-998, 1040-1051, 1080-1090, 1154-1168, 1215-1231, 1278-1302, 1378-1400, 1460-1469, 1497-1519, 1606-1626 and 1639-1651.

7. The antibody of claim 1, where the antibody is labeled.

8. An isolated Robo-specific antibody that binds to a protein of SEQ ID NO: 2, 4, 6, or 12.

9. An isolated Robo polypeptide comprising at least one immunoglobulin or fibronectin domain, wherein the polypeptide has least 25 consecutive residues of SEQ ID NO:8 or 10 and modulates Robo-mediated signaling; or an immunogenic Robo polypeptide comprising one or more sequences selected from the group consisting of:
(a) an immunogenic polypeptide of SEQ ID NO:8 selected from the group of residues 18-28, 31-40, 45-65, 106-116, 137-145, 174-184, 214-230, 274-286, 314-324, 399-412, 496-507, 548-565, 599-611, 660-671, 717-730, 780-791, 835-847, 877-891, 930-942, 981-998, 1040-1051, 1080-1090, 1154-1168, 1215-1231, 1278-1302, 1378-1400, 1460-1469, 1497-1519, 1606-1626 and 1639-1651 of SEQ ID NO:8; and
(b) an immunogenic polypeptide of SEQ ID NO:10 selected from the group of residues 5-16, 38-47, 83-94, 112-125, 168-180, 195-209, 222-235 and 241-254 of SEQ ID NO:10.

10. A method of identifying a compound that modulates Robo-mediated signaling comprising: incubating a test compound with a Robo polypeptide of claim 9; and determining a change in Robo-mediated signaling.

11. An isolated nucleic acid encoding a Robo polypeptide of claim 9.


SEQUENCE LISTING (13 sequences)

SEQ ID NO: 1 - DNA (cDNA), 4188 bp; double-stranded, linear
atgcatcccatgcatcccgaaaaccacgccatcgcccggagcacgagcaccactaataacccatctcgcagtcggagcagcaggatgtggctcctgcccgcctggctgctcctcgtcctggtggccagcaatggcctgccagcagtcagaggccagtaccaatcgccacgtatcatcgagcatcccacggatctggtcgttaagaagaatgaacccgccacgctcaactgcaaagtggagggcaagccggaacccaccattgagtggtttaaggatggcgaacccgtcagcaccaacgaaaagaaatcgcaccgcgtccagttcaaggacggcgccctcttcttttacaggacaatgcaaggcaagaaggagcaggacggcggagagtactggtgcgtggccaagaaccgagtgggccaggccgttagtcgccatgcctccctccagatagctgttttgcgcgacgattttcgcgtggagcccaaagacacgcgagtggccaaaggcgagacggctctgctggagtgtgggccgcccaaaggcattccagagccaacgctgatttggataaaggacggcgttcccttggacgacctgaaagccatgtcgtttggcgccagctcccgcgttcgaattgtggacggtggcaacctgctgatcagcaatgtggagcccattgatgagggcaactacaagtgcattgcccagaatctggtaggcacccgcgagagcagctatgccaagctgattgtccaggtcaaaccatactttatgaaggagcccaaggatcaggtgatgctctacggccagacagccactttccactgctcagtgggcggtgatccgccgccgaaagtgttgtggaaaaaggaggagggcaatattccggtgtccagagcgcgaatccttcacgacgagaaaagtttagagatatccaacataacgcccaccgatgagggcacctatgtctgcgaggcacacaacaatgtcggtcagatcagcgctagggcttctcttatagtccacgctccgccgaactttacgaaaagacccagtaacaagaaagtgggactaaatggggttgtccaactaccttgcatggcctccggaaaccctccgccgtctgtattctggaccaaggaaggagtatccactcttatgttcccaaatagttcgcacggaaggcagtatgtggctgccgatggaactctgcagattacggatgtgcggcaggaagacgaaggctactatgtgtgttccgctttcagtgtagtcgattcctctacagtacgggttttcctgcaagtcagctcggtagacgagcgtccacctccgattattcaaatcggacctgccaatcaaacactgcccaagggatcagttgctactttaccctgtcgggccactggaaatcccagtccccgtatcaagtggttccacgatggacatgccgtacaagcgggcaatcgatacagcatcatccaaggaagctcactgagagtcgatgaccttcaactaagtgactctggtacctacacctgcactgcatctggcgaacgaggagaaacttcctgggctgccacactaacggtggaaaaacccggttctacatctcttcaccgggcagctgatcctagcacttatcctgctcctccaggaacacctaaagtcctgaatgtcagtcgcaccagcattagtcttcgttgggctaaaagccaagagaaacccggagctgtgggcccaatcattggatacactgtagagtacttcagtccggatctgcaaactggttggattgtggctgcccatcgagtcggcgacactcaagtcactatctcgggtctcactcctggcacttcgtatgtgttcctagttagagctgagaatactcagggtatttctgtgccttccggcttatcaaatgttattaaaaccattgaggcagatttcgatgcagcttctgccaatgatttgtcagcagctcgaactttgctgacaggaaagtcggtggagctaatagatgcctcggctatcaatgctagtgccgttagacttgagtggatgctccacgtgagcgctgatgagaaatacgtagagggcctgcgcatacactataaggatgccagtgtaccatccgcacagtatcactcgatcactgttatggatgcctctgcagaatcgtttgtggtgggaaaccttaagaagtacaccaagtatgagttcttcctaacacccttttttgagacaattgaaggacagcccagtaactccaagacagccctcacctatgaagatgttccctccgcaccaccggataacattcagattggcatgtacaaccaaacagccggttgggtgcgttggactccgccaccctcccagcaccacaatggcaatttgtatggctacaagattgaggtcagcgccggtaacaccatgaaggtgctggccaatatgactcttaatgctaccaccacatctgtgctcctaaataacctaaccaccggagctgtgtacagcgtgaggttgaactcctttaccaaggcaggagatggaccttactccaaaccgatatcactattcatggaccccacccatcatgtgcatccgccacgggcacatccaagcggcacccatgatgggcgacatgagggacaggatctcacgtatcataacaatggcaacataccacctggcgacattaatcccaccactcataaaaagaccactgactacctatctggaccgtggctaatggtgctggtctgcatcgttcttctagtcctggttatttcggcggctatttcgatggtctacttcaagcgcaagcatcaaatgaccaaggaattgggtcacttaagtgtggtcagtgacaacgaaataaccgcattaaatatcaatagcaaagagagcctttggatagaccatcatcgtggatggcgaactgccgatactgacaaagactcaggattaagcgaatcgaagctactatcccacgttaacagcagtcaatccaactacaataactccgatggaggaaccgattatgcagaagttgacacccgtaaccttaccaccttctacaattgtcgcaagagccccgataatcccacgccgtacgccaccactatgatcattggtacctcttccagtgagacctgcaccaagacaacatctataagtgccgataaggactcgggaactcattcgccctattctgacgcatttgccggtcaggtgccagcggttcctgttgtcaaatccaactatcttcagtatccggttgaaccgatcaactggtcagagtttctacccccgccgccagaacacccacctccgtcttctacctatggatacgcacaaggatctcctgaatcttcgcggaagagctccaaaagcgcaggttccggcatttctacaaatcaaagcattctgaacgcatccatacacagcagctcctcgggcggcttttcagcttggggagtatcgccccaatatgctgtcgcctgtccaccggaaaacgtttatagcaatccgctgtcggcagtggctggcggcacccagaaccgctatcagataacgcccacaaaccaacatccgccacagttaccggcctactttgccaccacgggtccaggaggagctgtaccacccaaccacctgccatttgccacacagcgtcatgcagccagcgagtaccaggctggactgaatgcagcgcgatgtgcccaaagccgcgcctgcaacagctgcgatgccttggccacaccctcgcccatgcaacccccaccgccagttcccgtacccgagggctggtaccaaccggtgcatcccaatagccacccgatgcacccgacctcctccaaccaccagatctaccagtgctcctccgagtgctcggatcactcgaggagctcgcagagtcacaagcggcagctgcagctcgaggagcacggcagcagtgccaaacaacgcggaggacaccaccgtcgacgagccccggtggtgcagccgtgcatggagagcgagaacgagaacatgctggcggagtacgagcagcgccagtacaccagcgattgctgcaatagctcccgcgagggcgacacctgctcctgcagcgagggatcctgtctttacgccgaggcgggcgagccggcgcctcgtcaaatgactgctaagaacacctaa

SEQ ID NO: 2 - protein, 1395 aa
MHPMHPENHAIARSTSTTNNPSRSRSSRMWLLPAWLLLVLVASNGLPAVRGQYQSPRIIEHPTDLVVKKNEPATLNCKVEGKPEPTIEWFKDGEPVSTNEKKSHRVQFKDGALFFYRTMQGKKEQDGGEYWCVAKNRVGQAVSRHASLQIAVLRDDFRVEPKDTRVAKGETALLECGPPKGIPEPTLIWIKDGVPLDDLKAMSFGASSRVRIVDGGNLLISNVEPIDEGNYKCIAQNLVGTRESSYAKLIVQVKPYFMKEPKDQVMLYGQTATFHCSVGGDPPPKVLWKKEEGNIPVSRARILHDEKSLEISNITPTDEGTYVCEAHNNVGQISARASLIVHAPPNFTKRPSNKKVGLNGVVQLPCMASGNPPPSVFWTKEGVSTLMFPNSSHGRQYVAADGTLQITDVRQEDEGYYVCSAFSVVDSSTVRVFLQVSSVDERPPPIIQIGPANQTLPKGSVATLPCRATGNPSPRIKWFHDGHAVQAGNRYSIIQGSSLRVDDLQLSDSGTYTCTASGERGETSWAATLTVEKPGSTSLHRAADPSTYPAPPGTPKVLNVSRTSISLRWAKSQEKPGAVGPIIGYTVEYFSPDLQTGWIVAAHRVGDTQVTISGLTPGTSYVFLVRAENTQGISVPSGLSNVIKTIEADFDAASANDLSAARTLLTGKSVELIDASAINASAVRLEWMLHVSADEKYVEGLRIHYKDASVPSAQYHSITVMDASAESFVVGNLKKYTKYEFFLTPFFETIEGQPSNSKTALTYEDVPSAPPDNIQIGMYNQTAGWVRWTPPPSQHHNGNLYGYKIEVSAGNTMKVLANMTLNATTTSVLLNNLTTGAVYSVRLNSFTKAGDGPYSKPISLFMDPTHHVHPPRAHPSGTHDGRHEGQDLTYHNNGNIPPGDINPTTHKKTTDYLSGPWLMVLVCIVLLVLVISAAISMVYFKRKHQMTKELGHLSVVSDNEITALNINSKESLWIDHHRGWRTADTDKDSGLSESKLLSHVNSSQSNYNNSDGGTDYAEVDTRNLTTFYNCRKSPDNPTPYATTMIIGTSSSETCTKTTSISADKDSGTHSPYSDAFAGQVPAVPVVKSNYLQYPVEPINWSEFLPPPPEHPPPSSTYGYAQGSPESSRKSSKSAGSGISTNQSILNASIHSSSSGGFSAWGVSPQYAVACPPENVYSNPLSAVAGGTQNRYQITPTNQHPPQLPAYFATTGPGGAVPPNHLPFATQRHAASEYQAGLNAARCAQSRACNSCDALATPSPMQPPPPVPVPEGWYQPVHPNSHPMHPTSSNHQIYQCSSECSDHSRSSQSHKRQLQLEEHGSSAKQRGGHHRRRAPVVQPCMESENENMLAEYEQRQYTSDCCNSSREGDTCSCSEGSCLYAEAGEPAPRQMTAKNT

SEQ ID NO: 3 - DNA (cDNA), 4146 bp; double-stranded, linear
ggtgaaaatccacgcatcatcgagcatcccatggacacgacggtgccaaaaaatgatccatttacgtttaattgccaggccgagggcaatccaacaccaaccattcaatggtttaaggacggtcgcgaactgaagacggatacgggttcgcatcgcataatgctgcccgccgggggtctattctttctcaaggttatccactcacgtagagagagcgatgcgggcacttactggtgcgaggccaaaaacgagtttggagtggcacggtccaggaatgcaacgttgcaagtggcagttctccgcgacgaattccgtttggagccggcaaatacccgcgtggcccaaggcgaggtggccctgatggaatgcggtgccccccgaggatctccggagccgcaaatctcgtggcgcaagaacggccagaccctgaatcttgtcgggaacaagcggattcgcattgtcgacggtggcaatctggccatccaggaagcccgccaatcggacgacggacgctaccagtgtgtggtcaagaatgtggttggcacccgggagtcggccaccgcttttcttaaagtgcatgtacgtccattcctcatccgaggaccccagaatcagacggcggtggtgggcagctcggtggtcttccagtgccgcatcggaggcgatcccctgcctgatgtcctgtggcgacgcactgcctccggcggcaatatgccactgcgtaagttttcttggcttcattcagcttcaggtcgtgtgcacgtacttgaggaccgcagtctgaagctggacgacgttactctggaggacatgggcgagtacacttgcgaggcggacaatgcggtgggcggcatcacggccactggcatcctcaccgttcacgctccccccaaatttgtgatacgccccaagaatcagctggtggagatcggtgatgaagtgctgttcgagtgccaagcgaatggacatccccgaccaacgctctactggtcggtggagggcaacagctccctgctgctccccggctatcgggatggccgcatggaagtgaccctgacgcccgaggggcgctcggtgctctcgatagctcgatttgcccgtgaggattccggaaaggtggtcacttgcaacgccctgaacgccgtgggcagcgtcagcagtcggactgtggtcagtgtggatacgcaattcgagctgccaccgccgattatcgaacaggggcccgtgaatcaaacgttgcccgttaaatcaattgtggttctgccatgccgaactctgggcactccagtgccacaggtctcttggtacctggatggcatacccatcgatgtgcaggagcacgagcggcggaatctttcggacgctggagccttaaccatttcggatcttcagcgccacgaggatgaaggcttgtacacctgcgtggccagcaatcgcaacggaaaatcctcttggagtggttaccttcgtctggacaccccgacaaatccgaatatcaagttcttcagagccccagaactttccacctacccagggccgccaggaaaaccgcaaatggtggagaagggcgaaaattcggtgactctcagctggacgaggagcaacaaggtgggcggctccagtctggtgggctatgtaatcgagatgtttggcaaaaacgaaacggatggctgggtggctgtgggcactagggtgcaaaataccacgtttacccaaacgggtctgctgccgggtgtgaattacttctttctaattcgagccgagaactcccatggcttatcactgcccagtccgatgtcggaacccattacggtgggaacgcgctacttcaatagtggtctggatctgagcgaggctcgtgccagtctgctgtccggagatgttgtggagctgagcaacgccagtgtggtggactccactagcatgaaactcacctggcagatcatcaatggcaaatacgtcgagggcttctatgtctatgcgagacagttgccaaatccaatagtcaacaatccggcgcccgttactagcaataccaatccgctgctgggctctacatccacatccgcatccgcatccgcctcggcatcggcattgatttcgacaaagccaaatattgcagctgccggcaaacgtgatggggagacaaaccagagtggaggaggagctccgaccccactgaacaccaagtatcgcatgctaacgattctcaatggcggtggcgcctcatcctgcaccatcaccgggctcgtccagtacacgctgtatgaatttttcatcgtgccattttacaaatccgtcgagggcaagccgtcgaattcgcgcatcgctcgcacccttgaagatgttccctctgaggcaccatatggaatggaggctctgctgttgaactcctccgcggtcttcctcaaatggaaggcaccagaactcaaggatcggcatggtgttctcttgaactatcatgttatagtccgaggtattgacactgcccacaatttctcacgcattttgacaaatgtcaccatcgatgccgcttcgcctactctggttttggccaatctcaccgaaggcgtcatgtacaccgtgggcgtggcggccggaaataacgctggagttggtccttattgtgtcccagctactttgcgtttggatcccatcacaaagcgactcgatccgttcatcaatcagcgggaccatgttaacgatgtgctgacgcagccctggttcataatactcctgggcgccatcctggccgttcttatgctgtcctttggcgcaatggtctttgtgaagcgcaagcacatgatgatgaagcagtcggccctaaatacaatgcgtggcaatcacacgagcgacgtgctcaaaatgccgagtctatcggcgcgcaatggaaacggctactggctggactcctccaccggcggaatggtgtggcgtccctcgcccggcggcgactcgctggagatgcaaaaggatcacatcgccgactatgcgccggtctgcggtgcccccggttctccggccggcggtggcacctcttccggtggatccggtggcgcgggcagcggtgccagcggcggcgatgacattcatggaggacacggcagcgaacgcaatcagcagcggtacgtgggcgagtactccaacataccgaccgactatgcagaggtgtccagttttggcaaggcacccagcgagtatggtcggcatggcaacgcctccccggccccttatgccacctcttcgatcctgagtccccaccagcagcaacagcagcagcagccgcgttatcaacagcgaccagtgcccggctatgggctccagcgcccaatgcacccacactaccagcagcagcagcatcagcagcaacaggcgcagcagacgcaccagcaacaccaggctctccagcagcaccagcaactgccacccagcaacatctaccagcagatgtccaccaccagcgagatataccccacgaacacgggtccttcgcgctctgtctactctgagcagtattactaccccaaggacaagcagagacacatccacatcaccgagaacaagctgagcaactgccacacctatgaggcggctcctggcgccaagcagtcctcgccgatatcctcgcagttcgccagcgtgaggcggcagcagctgccgcccaactgcagcatcggcagggaaagtgcccgcttcaaggtgctaaacacggatcagggcaagaaccagcagaatctcctggatctcgacggctcctcgatgtgctacaacggtctggcagactcgggctgcggtggatctccctccccgatggccatgctgatgtcgcacgaggacgagcacgcgctgtaccacacggcggatggggatctggacgacatggaacgactgtacgtcaaggtggacgagcagcagcctccacagcagcagcagcagctgattcccctggtcccacagcatccggcggaaggtcacctgcagtcctggcggaatcagagcacgcggagcagtcggaagaacggccaggaatgcatcaaggaacccagcgagttgatctacgctccgggaagcgtggccagcgaacggagcctcctcagcaactcgggtagcggcaccagcagccagccagctggccacaatgtctga

SEQ ID NO: 4 - protein, 1381 aa
GENPRIIEHPMDTTVPKNDPFTFNCQAEGNPTPTIQWFKDGRELKTDTGSHRIMLPAGGLFFLKVIHSRRESDAGTYWCEAKNEFGVARSRNATLQVAVLRDEFRLEPANTRVAQGEVALMECGAPRGSPEPQISWRKNGQTLNLVGNKRIRIVDGGNLAIQEARQSDDGRYQCVVKNVVGTRESATAFLKVHVRPFLIRGPQNQTAVVGSSVVFQCRIGGDPLPDVLWRRTASGGNMPLRKFSWLHSASGRVHVLEDRSLKLDDVTLEDMGEYTCEADNAVGGITATGILTVHAPPKFVIRPKNQLVEIGDEVLFECQANGHPRPTLYWSVEGNSSLLLPGYRDGRMEVTLTPEGRSVLSIARFAREDSGKVVTCNALNAVGSVSSRTVVSVDTQFELPPPIIEQGPVNQTLPVKSIVVLPCRTLGTPVPQVSWYLDGIPIDVQEHERRNLSDAGALTISDLQRHEDEGLYTCVASNRNGKSSWSGYLRLDTPTNPNIKFFRAPELSTYPGPPGKPQMVEKGENSVTLSWTRSNKVGGSSLVGYVIEMFGKNETDGWVAVGTRVQNTTFTQTGLLPGVNYFFLIRAENSHGLSLPSPMSEPITVGTRYFNSGLDLSEARASLLSGDVVELSNASVVDSTSMKLTWQIINGKYVEGFYVYARQLPNPIVNNPAPVTSNTNPLLGSTSTSASASASASALISTKPNIAAAGKRDGETNQSGGGAPTPLNTKYRMLTILNGGGASSCTITGLVQYTLYEFFIVPFYKSVEGKPSNSRIARTLEDVPSEAPYGMEALLLNSSAVFLKWKAPELKDRHGVLLNYHVIVRGIDTAHNFSRILTNVTIDAASPTLVLANLTEGVMYTVGVAAGNNAGVGPYCVPATLRLDPITKRLDPFINQRDHVNDVLTQPWFIILLGAILAVLMLSFGAMVFVKRKHMMMKQSALNTMRGNHTSDVLKMPSLSARNGNGYWLDSSTGGMVWRPSPGGDSLEMQKDHIADYAPVCGAPGSPAGGGTSSGGSGGAGSGASGGDDIHGGHGSERNQQRYVGEYSNIPTDYAEVSSFGKAPSEYGRHGNASPAPYATSSILSPHQQQQQQQPRYQQRPVPGYGLQRPMHPHYQQQQHQQQQAQQTHQQHQALQQHQQLPPSNIYQQMSTTSEIYPTNTGPSRSVYSEQYYYPKDKQRHIHITENKLSNCHTYEAAPGAKQSSPISSQFASVRRQQLPPNCSIGRESARFKVLNTDQGKNQQNLLDLDGSSMCYNGLADSGCGGSPSPMAMLMSHEDEHALYHTADGDLDDMERLYVKVDEQQPPQQQQQLIPLVPQHPAEGHLQSWRNQSTRSSRKNGQECIKEPSELIYAPGSVASERSLLSNSGSGTSSQPAGHNV

SEQ ID NO: 5 - DNA (cDNA), 3894 bp; double-stranded, linear
atgtactatctaggtttttaccacactcacacacacacacacacatacataaattttgataaaattcctaatgcctcaaatctcgctcccgtgataatcgaacatcccatcgatgtggtggtatctaggggatcgccagcaaccctcaactgtggtgcaaagccatctaccgccaaaatcacatggtacaaggatggacagcccgtaatcacgaataaggagcaagtgaacagccaccggattgttctcgacacgggatccctgtttcttctgaaagtgaatagtggaaaaaacggaaaagacagcgatgcgggagcgtactattgtgtggccagcaacgagcacggagaagtgaagtcgaacgaaggatcgttaaaattggcgatgcttcgcgaagactttcgagttcggccaagaacagttcaggctcttggtggagagatggccgttctggaatgcagtccgccacgtggattcccggagccggttgtgagctggcggaaagacgacaaagagctccgaattcaagacatgccacgatacactctacactctgacggaaacctcatcattgatccggtcgatcgaagcgattctggtacttatcagtgtgttgccaacaacatggtcggagaacgggtgtccaatcccgcaagattgagtgtctttgagaaaccaaagtttgagcaagaacccaaggacatgacggtcgacgtcggagccgcagtgctgtttgattgtcgtgtgactggagatcctcaaccacaaattacgtggaaacgcaaaaatgagccgatgccagttacacgtgcatacattgccaaggataatcgggggttgagaatcgaaagagttcaaccatcagacgaaggtgaatacgtttgctatgcacgaaatccagcgggaactcttgaagcatctgcacatcttcgtgtccaggcacctccatccttccagacaaaaccagcagaccagtcagttccagctggaggcacggcaacttttgaatgcaccttggtcggtcaaccgagtcccgcctatttttggagcaaggaaggccaacaggatcttcttttcccaagttatgtgtccgctgatggtagaacgaaagtttcaccaactggaacattgacaattgaggaagttcgtcaagttgatgagggagcttatgtgtgcgctggaatgaactcggcaggaagctcgttgagcaaggcagctttgaaagcaacatttgaaaccaaaggccgtgtccaaaaaaaaaagagcaaaatgggcaaacagaaacaaaaaaatgttcaatcaattatcaaatatttaatttcagccgtgaccggaaacacacccgccaaaccaccaccaacaatcgagcatggtcatcaaaatcagacccttatggttggatcatcagccatccttccatgtcaggctagcggaaaaccaactccaggaatatcatggctcagggatgggctacctattgacattacagatagtcgtatcagtcaacattcaacgggaagtctacatattgccgatttaaagaaacctgacaccggagtttacacttgcattgcgaagaacgaggatggagagtcaacatggtcggcatctctgactgttgaagatcacactagcaatgcacaatttgttcggatgccggatccatcgaacttcccgtcttctccaacgcaacccattattgtcaatgtcactgataccgaagtagagctccactggaatgctccctccacatctggcgcaggaccaatcactggttatatcattcagtactacagtccagacctcggacagacgtggtttaacattccagactacgtggcatctactgaatatagaataaagggtctgaaaccatctcactcgtatatgtttgtgattcgagcagaaaatgagaaaggtattggaacgccgagtgtgtcgtcggctctcgttaccactagcaagccagcagctcaagttgcgctttctgacaagaacaaaatggacatggccatcgctgagaagagactcacttcggaacaactcataaaactcgaggaagtgaagactattaattctacggccgttcgtttgttctggaagaagaggaaacttgaagagctgattgatggttactacatcaagtggagagggcctccaagaaccaatgataatcaatacgtgaatgtgaccagccctagcaccgaaaactatgttgtttcaaatttaatgccattcaccaactatgagtttttcgtgattccttatcattccggagttcatagtattcatggagcaccgagtaattccatggacgtgttgaccgccgaagctccaccttcattgccaccagaggatgtgcgaatccgtatgctcaacctgaccactcttcgtatctcttggaaagcaccaaaagccgacggcatcaacggaattctcaaaggattccaaattgttattgttggtcaagcgcccaacaacaatcggaacatcactacaaacgagagagctgccagtgttactctgttccatttagtgactggaatgacgtataaaattcgtgtagcggctagaagcaatggtggagttggagtctcacatggaacgagtgaagtcatcatgaatcaagacacgctggaaaaacaccttgctgctcaacaagaaaacgaatcatttttgtatgggctgatcaataaatctcatgttcctgtgattgtcattgttgcaattctgattattttcgtagtcatcattatagcctattgttactggaggaatagcagaaacagtgatggaaaggatcgaagttttataaagatcaatgatggaagtgttcatatggcttcgaataatctttgggatgttgcacaaaatccgaatcagaatccaatgtacaacactgctggaagaatgactatgaacaatagaaatggccaggctctctattcgctgacaccaaatgcgcaagactttttcaacaattgtgatgactacagtggaacgatgcacagaccaggatccgagcatcactatcattatgctcaactgactggcggacctggtaatgcgatgtctactttttatggaaaccaatatcacgatgatccatctccatatgccaccacaacactggtcctgtcgaaccaacaaccagcttggctcaatgacaaaatgcttcgcgcgccagcaatgccaacaaatcccgtgccaccagagccaccggcgcgatatgcagatcataccgctggaagacgatctcgatcgagccgtgcatccgatgggagaggaactctgaatggcggactccatcaccggactagcggaagtcaacggtcggatagtccacctcacacagatgtgagctatgttcagcttcactcatccgatggaactggtagtagtaaggaaagaactggggagcggagaacaccaccgaataagactctgatggactttattccgccaccaccttccaatccaccaccacctggagggcacgtttatgacacagcaactaggcgtcagttgaatcgtggaagtactccacgagaagacacctacgattcggtcagtgacggagcttttgctcgggttgatgtgaatgcaaggccaacgagtcggaatcggaatttgggaggaaggccgctgaaagggaaacgagacgacgatagtcagcggtcttcgttgatgatggacgatgatggtggatcttctgaagctgacggggagaactctgaaggagacgttccgcgtggaggtgttagaaaagcagttcctcgaatgggtatctctgcaagtacgctggctcatagttgttacgggacaaacggcactgctcaacgattccggtcaattccacgtaacaatggaatcgtcacacaagaacaaacttga

SEQ ID NO: 6 - protein, 1297 aa
MYYLGFYHTHTHTHTYINFDKIPNASNLAPVIIEHPIDVVVSRGSPATLNCGAKPSTAKITWYKDGQPVITNKEQVNSHRIVLDTGSLFLLKVNSGKNGKDSDAGAYYCVASNEHGEVKSNEGSLKLAMLREDFRVRPRTVQALGGEMAVLECSPPRGFPEPVVSWRKDDKELRIQDMPRYTLHSDGNLIIDPVDRSDSGTYQCVANNMVGERVSNPARLSVFEKPKFEQEPKDMTVDVGAAVLFDCRVTGDPQPQITWKRKNEPMPVTRAYIAKDNRGLRIERVQPSDEGEYVCYARNPAGTLEASAHLRVQAPPSFQTKPADQSVPAGGTATFECTLVGQPSPAYFWSKEGQQDLLFPSYVSADGRTKVSPTGTLTIEEVRQVDEGAYVCAGMNSAGSSLSKAALKATFETKGRVQKKKSKMGKQKQKNVQSIIKYLISAVTGNTPAKPPPTIEHGHQNQTLMVGSSAILPCQASGKPTPGISWLRDGLPIDITDSRISQHSTGSLHIADLKKPDTGVYTCIAKNEDGESTWSASLTVEDHTSNAQFVRMPDPSNFPSSPTQPIIVNVTDTEVELHWNAPSTSGAGPITGYIIQYYSPDLGQTWFNIPDYVASTEYRIKGLKPSHSYMFVIRAENEKGIGTPSVSSALVTTSKPAAQVALSDKNKMDMAIAEKRLTSEQLIKLEEVKTINSTAVRLFWKKRKLEELIDGYYIKWRGPPRTNDNQYVNVTSPSTENYVVSNLMPFTNYEFFVIPYHSGVHSIHGAPSNSMDVLTAEAPPSLPPEDVRIRMLNLTTLRISWKAPKADGINGILKGFQIVIVGQAPNNNRNITTNERAASVTLFHLVTGMTYKIRVAARSNGGVGVSHGTSEVIMNQDTLEKHLAAQQENESFLYGLINKSHVPVIVIVAILIIFVVIIIAYCYWRNSRNSDGKDRSFIKINDGSVHMASNNLWDVAQNPNQNPMYNTAGRMTMNNRNGQALYSLTPNAQDFFNNCDDYSGTMHRPGSEHHYHYAQLTGGPGNAMSTFYGNQYHDDPSPYATTTLVLSNQQPAWLNDKMLRAPAMPTNPVPPEPPARYADHTAGRRSRSSRASDGRGTLNGGLHHRTSGSQRSDSPPHTDVSYVQLHSSDGTGSSKERTGERRTPPNKTLMDFIPPPPSNPPPPGGHVYDTATRRQLNRGSTPREDTYDSVSDGAFARVDVNARPTSRNRNLGGRPLKGKRDDDSQRSSLMMDDDGGSSEADGENSEGDVPRGGVRKAVPRMGISASTLAHSCYGTNGTAQRFRSIPRNNGIVTQEQT

SEQ ID NO: 7 - DNA (cDNA), 4956 bp; double-stranded, linear
atgaaatggaaacatgttccttttttggtcatgatatcactcctcagcttatccccaaatcacctgtttctggcccagcttattccagaccctgaagatgtagagagggggaacgaccacgggacgccaatccccacctctgataacgatgacaattcgctgggctatacaggctcccgtcttcgtcaggaagattttccacctcgcattgttgaacacccttcagacctgattgtctcaaaaggagaacctgcaactttgaactgcaaagctgaaggccgccccacacccactattgaatggtacaaagggggagagagagtggagacagacaaagatgaccctcgctcacaccgaatgttgctgccgagtggatctttatttttcttacgtatagtacatggacggaaaagtagacctgatgaaggagtctatgtctgtgtagcaaggaattaccttggagaggctgtgagccacaatgcatcgctggaagtagccatacttcgggatgacttcagacaaaacccttcggatgtcatggttgcagtaggagagcctgcagtaatggaatgccaacctccacgaggccatcctgagcccaccatttcatggaagaaagatggctctccactggatgataaagatgaaagaataactatacgaggaggaaagctcatgatcacttacacccgtaaaagtgacgctggcaaatatgtttgtgttggtaccaatatggttggggaacgtgagagtgaagtagccgagctgactgtcttagagagaccatcatttgtgaagagacccagtaacttggcagtaactgtggatgacagtgcagaatttaaatgtgaggcccgaggtgaccctgtacctacagtacgatggaggaaagatgatggagagctgcccaaatccagatatgaaatccgagatgatcataccttgaaaattaggaaggtgacagctggtgacatgggttcatacacttgtgttgcagaaaatatggtgggcaaagctgaagcatctgctactctgactgttcaagaacctccacattttgttgtgaaaccccgtgaccaggttgttgctttgggacggactgtaacttttcagtgtgaagcaaccggaaatcctcaaccagctattttctggaggagagaagggagtcagaatctacttttctcatatcaaccaccacagtcatccagccgattttcagtctcccagactggcgacctcacaattactaatgtccagcgatctgatgttggttattacatctgccagactttaaatgttgctggaagcatcatcacaaaggcatatttggaagttacagatgtgattgcagatcggcctcccccagttattcgacaaggtcctgtgaatcagactgtagccgtggatggcactttcgtcctcagctgtgtggccacaggcagtccagtgcccaccattctgtggagaaaggatggagtcctcgtttcaacccaagactctcgaatcaaacagttggagaatggagtactgcagatccgatatgctaagctgggtgatactggtcggtacacctgcattgcatcaacccccagtggtgaagcaacatggagtgcttacattgaagttcaagaatttggagttccagttcagcctccaagacctactgacccaaatttaatccctagtgccccatcaaaacctgaagtgacagatgtcagcagaaatacagtcacattatcgtggcaaccaaatttgaattcaggagcaactccaacatcttatattatagaagccttcagccatgcatctggtagcagctggcagaccgtagcagagaatgtgaaaacagaaacatctgccattaaaggactcaaacctaatgcaatttaccttttccttgtgagggcagctaatgcatatggaattagtgatccaagccaaatatcagatccagtgaaaacacaagatgtcctaccaacaagtcagggggtggaccacaagcaggtccagagagagctgggaaatgctgttctgcacctccacaaccccaccgtcctttcttcctcttccatcgaagtgcactggacagtagatcaacagtctcagtatatacaaggatataaaattctctatcggccatctggagccaaccacggagaatcagactggttagtttttgaagtgaggacgccagccaaaaacagtgtggtaatccctgatctcagaaagggagtcaactatgaaattaaggctcgccctttttttaatgaatttcaaggagcagatagtgaaatcaagtttgccaaaaccctggaagaagcacccagtgccccaccccaaggtgtaactgtatccaagaatgatggaaacggaactgcaattctagttagttggcagccacctccagaagacactcaaaatggaatggtccaagagtataaggtttggtgtctgggcaatgaaactcgataccacatcaacaaaacagtggatggttccaccttttccgtggtcattccctttcttgttcctggaatccgatacagtgtggaagtggcagccagcactggggctgggtctggggtaaagagtgagcctcagttcatccagctggatgcccatggaaaccctgtgtcacctgaggaccaagtcagcctcgctcagcagatttcagatgtggtgaagcagccggccttcatagcaggtattggagcagcctgttggatcatcctcatggtcttcagcatctggctttatcgacaccgcaagaagagaaacggacttactagtacctacgcgggtatcagaaaagtcccgtcttttaccttcacaccaacagtaacttaccagagaggaggcgaagctgtcagcagtggagggaggcctggacttctcaacatcagtgaacctgccgcgcagccatggctggcagacacgtggcctaatactggcaacaaccacaatgactgctccatcagctgctgcacggcaggcaatggaaacagcgacagcaacctcactacctacagtcgcccagctgattgtatagcaaattataacaaccaactggataacaaacaaacaaatctgatgctccctgagtcaactgtttatggtgatgtggaccttagtaacaaaatcaatgagatgaaaaccttcaatagcccaaatctgaaggatgggcgttttgtcaatccatcagggcagcctactccttacgccaccactcagctcatccagtcaaacctcagcaacaacatgaacaatggcagcggggactctggcgagaagcactggaaaccactgggacagcagaaacaagaagtggcaccagttcagtacaacatcgtggagcaaaacaagctgaacaaagattatcgagcaaatgacacagttcctccaactatcccatacaaccaatcatacgaccagaacacaggaggatcctacaacagctcagaccggggcagtagtacatctgggagtcaggggcacaagaaaggggcaagaacacccaaggtaccaaaacagggtggcatgaactgggcagacctgcttcctcctcccccagcacatcctcctccacacagcaatagcgaagagtacaacatttctgtagatgaaagctatgaccaagaaatgccatgtcccgtgccaccagcaaggatgtatttgcaacaagatgaattagaagaggaggaagatgaacgaggccccactccccctgttcggggagcagcttcttctccagctgccgtgtcctatagccatcagtccactgccactctgactccctccccacaggaagaactccagcccatgttacaggattgtccagaggagactggccacatgcagcaccagcccgacaggagacggcagcctgtgagtcctcctccaccaccacggccgatctcccctccacatacctatggctacatttcaggacccctggtctcagatatggatacggatgcgccagaagaggaagaagacgaagccgacatggaggtagccaagatgcaaaccagaaggcttttgttacgtgggcttgagcagacacctgcctccagtgttggggacctggagagctctgtcacggggtccatgatcaacggctggggctcagcctcagaggaggacaacatttccagcggacgctccagtgttagttcttcggacggctcctttttcactgatgctgactttgcccaggcagtcgcagcagcggcagagtatgctggtctgaaagtagcacgacggcaaatgcaggatgctgctggccgtcgacattttcatgcgtctcagtgccctaggcccacaagtcccgtgtctacagacagcaacatgagtgccgccgtaatgcagaaaaccagaccagccaagaaactgaaacaccagccaggacatctgcgcagagaaacctacacagatgatcttccaccacctcctgtgccgccacctgctataaagtcacctactgcccaatccaagacacagctggaagtacgacctgtagtggtgccaaaactcccttctatggatgcaagaacagacagatcatcagacagaaaaggaagcagttacaaggggagagaagtgttggatggaagacaggttgttgacatgcgaacaaatccaggtgatcccagagaagcacaggaacagcaaaatgacgggaaaggacgtggaaacaaggcagcaaaacgagaccttccaccagcaaagactcatctcatccaagaggatattctaccttattgtagacctacttttccaacatcaaataatcccagagatcccagttcctcaagctcaatgtcatcaagaggatcaggaagcagacaaagagaacaagcaaatgtaggtcgaagaaatattgcagaaatgcaggtacttggaggatatgaaagaggagaagataataatgaagaattagaggaaactgaaagctga

SEQ ID NO: 8 - protein, 1651 aa
MKWKHVPFLVMISLLSLSPNHLFLAQLIPDPEDVERGNDHGTPIPTSDNDDNSLGYTGSRLRQEDFPPRIVEHPSDLIVSKGEPATLNCKAEGRPTPTIEWYKGGERVETDKDDPRSHRMLLPSGSLFFLRIVHGRKSRPDEGVYVCVARNYLGEAVSHNASLEVAILRDDFRQNPSDVMVAVGEPAVMECQPPRGHPEPTISWKKDGSPLDDKDERITIRGGKLMITYTRKSDAGKYVCVGTNMVGERESEVAELTVLERPSFVKRPSNLAVTVDDSAEFKCEARGDPVPTVRWRKDDGELPKSRYEIRDDHTLKIRKVTAGDMGSYTCVAENMVGKAEASATLTVQEPPHFVVKPRDQVVALGRTVTFQCEATGNPQPAIFWRREGSQNLLFSYQPPQSSSRFSVSQTGDLTITNVQRSDVGYYICQTLNVAGSIITKAYLEVTDVIADRPPPVIRQGPVNQTVAVDGTFVLSCVATGSPVPTILWRKDGVLVSTQDSRIKQLENGVLQIRYAKLGDTGRYTCIASTPSGEATWSAYIEVQEFGVPVQPPRPTDPNLIPSAPSKPEVTDVSRNTVTLSWQPNLNSGATPTSYIIEAFSHASGSSWQTVAENVKTETSAIKGLKPNAIYLFLVRAANAYGISDPSQISDPVKTQDVLPTSQGVDHKQVQRELGNAVLHLHNPTVLSSSSIEVHWTVDQQSQYIQGYKILYRPSGANHGESDWLVFEVRTPAKNSVVIPDLRKGVNYEIKARPFFNEFQGADSEIKFAKTLEEAPSAPPQGVTVSKNDGNGTAILVSWQPPPEDTQNGMVQEYKVWCLGNETRYHINKTVDGSTFSVVIPFLVPGIRYSVEVAASTGAGSGVKSEPQFIQLDAHGNPVSPEDQVSLAQQISDVVKQPAFIAGIGAACWIILMVFSIWLYRHRKKRNGLTSTYAGIRKVPSFTFTPTVTYQRGGEAVSSGGRPGLLNISEPAAQPWLADTWPNTGNNHNDCSISCCTAGNGNSDSNLTTYSRPADCIANYNNQLDNKQTNLMLPESTVYGDVDLSNKINEMKTFNSPNLKDGRFVNPSGQPTPYATTQLIQSNLSNNMNNGSGDSGEKHWKPLGQQKQEVAPVQYNIVEQNKLNKDYRANDTVPPTIPYNQSYDQNTGGSYNSSDRGSSTSGSQGHKKGARTPKVPKQGGMNWADLLPPPPAHPPPHSNSEEYNISVDESYDQEMPCPVPPARMYLQQDELEEEEDERGPTPPVRGAASSPAAVSYSHQSTATLTPSPQEELQPMLQDCPEETGHMQHQPDRRRQPVSPPPPPRPISPPHTYGYISGPLVSDMDTDAPEEEEDEADMEVAKMQTRRLLLRGLEQTPASSVGDLESSVTGSMINGWGSASEEDNISSGRSSVSSSDGSFFTDADFAQAVAAAAEYAGLKVARRQMQDAAGRRHFHASQCPRPTSPVSTDSNMSAAVMQKTRPAKKLKHQPGHLRRETYTDDLPPPPVPPPAIKSPTAQSKTQLEVRPVVVPKLPSMDARTDRSSDRKGSSYKGREVLDGRQVVDMRTNPGDPREAQEQQNDGKGRGNKAAKRDLPPAKTHLIQEDILPYCRPTFPTSNNPRDPSSSSSMSSRGSGSRQREQANVGRRNIAEMQVLGGYERGEDNNEELEETES

SEQ ID NO: 9 - DNA (cDNA), 1300 bp; double-stranded, linear
  misc_feature 855..1187: /note= “N signifies gap in sequence”
cagattgttgctcaaggtcgaacagtgacatttccctgtgaaactaaaggaaacccacagccagctgttttttggcagaaagaaggcagccagaacctacttttcccaaaccaaccccagcagcccaacagtagatgctcagtgtcaccaactggagacctcacaatcaccaacattcaacgttccgacgcgggttactacatctgccaggctttaactgtggcaggaagcattttagcaaaagctcaactggaggttactgatgttttgacagatagacctccacctataattctacaaggcccagccaaccaaacgctggcagtggatggtacagcgttactgaaatgtaaagccactggtgatcctcttcctgtaattagctggttaaaggagggatttacttttccgggtagagatccaagagcaacaattcaagagcaaggcacactgcagattaagaatttacggatttctgatactggcacttatacttgtgtggctacaagttcaagtggagaggcttcctggagtgcagtgctggatgtgacagagtctggagcaacaatcagtaaaaactatgatttaagtgacctgccagggccaccatccaaaccgcaagtcactgatgttactaagaacagtgtcaccttgtcctggcagccaggtacccctggaacccttccagcaagtgcatatatcattgaggctttcagccaatcagtgagcaacagctggcagaccgtggcaaaccatgtaaagaccaccctctatactgtaagaggactgcggcccaatacaatctacttattcatggtcagagcgatcaaccccaaggtytcagtgacccaagtnaaaccacagaaaaacaatggatccacttgggccaatgtccctctacctccccccccagtccagccccttcctggcacggagctggaacactatgcagtggaacaacaagaaaatggctatgacagtgatagctggtgcccaccattgccagtacaaacttacttacaccaaggtctggaagatgaactggaagaagatgatgatagggtcccaacacctcctgttcgaggcgtggcttcttctcctgctatctcctttggacagcagtccactgcaactcttactccatccccacgggaagagatgcaacccatgctgcaggcttcacctntttacctcctctcaaagacctcgacctaccagcccattttctactgacagtaacaccagtgcagccctgagtcaaagtcagaggcctcggcccactaaaaaacacaagggaggg

SEQ ID NO: 10 - protein, 434 aa
  Modified-site 285..396: /note= “Xaa signifies gap in sequence”
QIVAQGRTVTFPCETKGNPQPAVFWQKEGSQNLLFPNQPQQPNSRCSVSPTGDLTITNIQRSDAGYYICQALTVAGSILAKAQLEVTDVLTDRPPPIILQGPANQTLAVDGTALLKCKATGDPLPVISWLKEGFTFPGRDPRATIQEQGTLQIKNLRISDTGTYTCVATSSSGEASWSAVLDVTESGATISKNYDLSDLPGPPSKPQVTDVTKNSVTLSWQPGTPGTLPASAYIIEAFSQSVSNSWQTVANHVKTTLYTVRGLRPNTIYLFMVRAINPKVSVTQXKPQKNNGSTWANVPLPPPPVQPLPGTELEHYAVEQQENGYDSDSWCPPLPVQTYLHQGLEDELEEDDDRVPTPPVRGVASSPAISFGQQSTATLTPSPREEMQPMLQASPXFTSSQRPRPTSPFSTDSNTSAALSQSQRPRPTKKHKGG

SEQ ID NO: 11 - DNA (cDNA), 444 bp; double-stranded, linear
gcccaggcagttgctgcagctgcggagtatgcgggcctgaaagtggctcgccgccaaatgcaagatgctgctggccgccgccacttccatgcctctcagtgcccaaggcccacgagtcctgtgtccacagacagcaacatgagtgctgttgtgatccagaaagccagacccgccaagaagcagaaacaccagccaggacatctgcgcagggaagcctacgcagatgatcttccaccccctccagtgccaccacctgctataaaatcgcccactgtccagtccaaggcacagctggaggtacggcctgtcatggtgccaaaactcgcgtctatagaagcaaggacagatagatcgtcagacagaaaaggaggcagttacaaggggagagaagctctggatggaagacaagtcactgacctgcgaacaaatccaagtgaccccaga

SEQ ID NO: 12 - protein, 148 aa
AQAVAAAAEYAGLKVARRQMQDAAGRRHFHASQCPRPTSPVSTDSNMSAVVIQKARPAKKQKHQPGHLRREAYADDLPPPPVPPPAIKSPTVQSKAQLEVRPVMVPKLASIEARTDRSSDRKGGSYKGREALDGRQVTDLRTNPSDPR

SEQ ID NO: 13 - protein, 909 aa
MFNRKTLLCTILLVLQAVIRSFCEDASNLAPVIIEHPIDVVVSRGSPATLNCGAKPSTAKITWYKDGQPVITNKEQVNSHRIVLDTGSLFLLKVNSGKNGKDSDAGAYYCVASNEHGEVKSNEGSLKLAMLREDFRVRPRTVQALGGEMAVLECSPPRGFPEPVVSWRKDDKELRIQDMPRYTLHSDGNLIIDPVDRSDSGTYQCVANNMVGERVSNPARLSVFEKPKFEQEPKDMTVDVGAAVLFDCRVTGDPQPQITWKRKNEPMPVTRAYIAKDNRGLRIERVQPSDEGEYVCYARNPAGTLEASAHLRVQAPPSFQTKPADQSVPAGGTATFECTLVGQPSPAYFWSKEGQQDLLFPSYVSADGRTKVSPTGTLTIEEVRQVDEGAYVCAGMNSAGSSLSKAALKVTTKAVTGNTPAKPPPTIEHGHQNQTLMVGSSAILPCQASGKPTPGISWLRDGLPIDITDSRISQHSTGSLHIADLKKPDTGVYTCIAKNEDGESTWSASLTVEDHTSNAQFVRMPDPSNFPSSPTQPIIVNVTDTEVELHWNAPSTSGAGPITGYIIQYYSPDLGQTWFNIPDYVASTEYRIKGLKPSHSYMFVIRAENEKGIGTPSVSSALVTTSKPAAQVALSDKNKMDMAIAEKRLTSEQLIKLEEVKTINSTAVRLFWKKRKLEELIDGYYIKWRGPPRTNDNQYVNVTSPSTENYVVSNLMPFTNYEFFVIPYHSGVHSIHGAPSNSMDVLTAEAPPSLPPEDVRIRMLNLTTLRISWKAPKADGINGILKGFQIVIVGQAPNNNRNITTNERAASVTLFHLVTGMTYKIRVAARSNGGVGVSHGTSEVIMNQDTLEKHLAAQQENESFLYGLINKSHVPVIVIVAILIIFVVIIIAYCYWRNSRNSDGKDRSF